(19) United States
(12) Patent Application Publication
(10) Pub. No.: US 2017/0138944 A1
(43) Pub. Date: May 18, 2017

(54) METHOD FOR DETECTING ENZYME ACTIVITY HYDROLYZING BETA-LACTAM RING ANTIMICROBIAL AGENTS

(71) Applicants: UNIVERSITÉ CATHOLIQUE DE LOUVAIN, Louvain la Neuve (BE); CENTRE HOSPITALIER UNIVERSITAIRE DINANT-GODINNE/UCL NAMUR, Dinant (BE)

(72) Inventors: Pierre BOGAERTS, Haltinne (BE); Sami YUNUS, Namur (BE)

(21) Appl. No.: 15/319,839

(22) PCT Filed: Jun. 19, 2015
(86) PCT No.: PCT/EP2015/063884

§ 371 Date: Dec. 19, 2016

(30) Foreign Application Priority Data
Jun. 19, 2014 (EP) 14173161.2

(51) Int. Cl. G01N 33/569; G01N 27/327; G01N 27/02

(52) U.S. Cl. CPC G01N 33/56911; G01N 27/02; G01N 27/3276; G01N 2333/986

(57) ABSTRACT

The present invention relates to a method for detecting an enzyme activity capable of hydrolyzing betalactam ring anti-microbial agents in a biological cell, comprising contacting said biological cell with at least one substrate of said enzyme activity comprising a betalactam ring, in an electrochemical cell, and detecting an impedance variation in said electrochemical cell with monitoring means. The present invention is in particular useful for detecting carbapenemase-producing Enterobacteriaceae (CPE).

### FIELD OF INVENTION

The present invention pertains to the field of detection of resistance to antibiotics. Especially, this invention relates to a method for detecting a beta-lactamase activity, comprising contacting a sample suspected to contain said enzyme activity with at least one substrate comprising a beta-lactam ring, in an electrochemical cell, and measuring impedance parameters in the electrochemical cell. The present invention is in particular useful for detecting carbapenemase-producing Enterobacteriaceae (CPE).

### BACKGROUND OF INVENTION

There is an ongoing need for sensors and methods for rapidly detecting a beta-lactamase activity in a biological cell, in particular for diagnosing pathogen agents responsible for an infection, or for detecting drug resistant pathogens.

Carbapenems currently represent the drugs of choice to the treatment of serious infections caused by multidrug-resistant Enterobacteriaceae strains producing Extended-Spectrum Beta-Lactamases (ESBLs). The recent emergence of carbapenem resistance has been increasingly reported among Enterobacteriaceae and now represents a major clinical concern. The most common mechanism of carbapenem resistance or decreased susceptibility in Enterobacteriaceae involves the presence of a beta-lactamase (a cephalosporinase or an ESBL) with low carbapenem-hydrolyzing activity together with decreased permeability due to porin loss or alteration. Carbapenem resistance also results more and more frequently from the production of carbapenemases belonging to Ambler Class A (mostly KPC), B (mostly VIM, IMP, NDM), or D (mostly OXA-48).

Considering the risk of spreading of carbapenems resistance, there is therefore a strong need for rapid methods and devices allowing carbapenemase-producing Enterobacteriaceae (CPE) to be detected with a high degree of confidence and a high specificity.

Currently, the complete identification of such a pathogen takes up to 72 hours in clinical laboratories, and includes the establishment of a resistance profile followed by confirmatory testing for the presence of a carbapenemase by phenotypic testing or molecular methods.

Molecular identification of the carbapenemases genes is limited since it only allows specific bacteria and/or resistance genes to be detected, and is further particularly expensive since it requires specific instruments, consumables, skilled personal and often dedicated rooms.

Currently the majority of the methods used to detect carbapenemases-producing organisms are based on phenotypic and genotypic methods.

The reference phenotypic method consists in determining qualitatively or quantitatively the resistance of a suspected pathogen against an antimicrobial agent by growing the said suspected pathogen in the presence of defined concentrations of this antimicrobial agent on solid agar plate or in liquid culture medium incubated for several hours. A confirmatory test necessitating 24 more hours of culture is then performed in order to determine if the resistance to the carbapenem is due to a carbapenemase or to another mechanism (disc combination test, Etest®, “Hodge-test”). Such methods are time consuming and lack sensitivity and specificity.

Several more straightforward methods were also developed, which comprise methods relying on the direct beta-lactam hydrolysis observation by beta-lactamases produced by resistant strains. Imipenem hydrolysis by a beta-lactamase of the type carbapenemase is represented below:

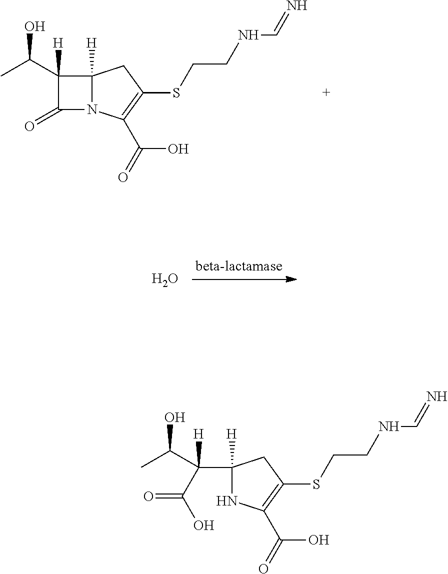 [SMILES: C[C@@H](O)[C@H]1C(=O)N2C(C(=O)O)=C(SCCNC=N)C[C@H]12 ; O ; C[C@@H](O)[C@@H](C(=O)O)[C@H]1CC(SCCNC=N)=C(C(=O)O)N1]

The hydrolysis of carbapenems, and more specifically of imipenem, can be monitored using intrinsic or extrinsic colorimetric methods. A method is qualified as intrinsic when the indicator is a sub-molecular part of the beta-lactam, and as extrinsic when the indicator is simply another reagent added together with a chosen beta-lactam.

Among the existing intrinsic methods, the betaLACTA™ test (Bio-Rad, Marnes-la-Coquette, France) relies on a chromogenic cephalosporin HMRZ-86, whose photochemical properties are strongly dependent on the beta-lactam ring opening by a beta-lactamase. The hydrolyzed molecule turns from yellow to red and can be detected by the naked eye. However, such chromogenic molecules cannot detect OXA-48 carbapenemase.

Recently, an extrinsic colorimetric method for the specific detection of carbapenemase activity was developed by Nordmann, Poirel and Dortet, which is referred to as the “CarbaNP-test”. This test particularly relies on the observation that imipenem hydrolysis results in the opening of its beta-lactam ring with the formation of one more carboxylic acid function. The resulting acidity increase can then be simply monitored by phenol red which acts as an acid-base colorimetric indicator. Phenol red change in color is then estimated by the naked eye. This method permits the detection of a carbapenemase-producing organism in about 3 hours and aims to eliminate the need of using phenotypic/genotypic confirmatory tests requiring an additional delay for being interpreted. However, the CarbaNP-test requires the pH color indicator and different reagents to be prepared, and necessitates a preliminary lysis procedure to liberate the beta-lactamase in the reaction medium. In addition, according to several studies, the CarbaNP-test still lacks sensitivity for the detection of OXA-48-producing organisms. Another drawback of this method is the fact that it relies on the naked eye operator's appreciation, which is subjective, especially when the color is not frankly yellow but orange instead, and is not easily traceable in the current Laboratory Information Management System (LIMS) of clinical laboratories.

The present inventors have surprisingly discovered that a new and original extrinsic method for identifying a beta-lactamase activity, and more specifically capable of identifying CPE, addresses the drawbacks encountered with the detection tests known in the art. The method of the present invention (also referred to hereinafter as the “BYG-test”, for Bogaerts-Yunus-Glupczynski), is advantageously faster, traceable, reusable more sensitive and specific and requires less material than the known detection tests. Further, in a specific embodiment, the method of the present invention allows the beta-lactamase activity to be detected directly in the biological cells containing it, and does not even require the tested biological cells (e.g. bacteria) to be lysed. The method of the present invention thus provides a fast, reliable and affordable solution for detecting any type of beta-lactamase producers, including producers of carbapenemase and/or of cephalosporinase, which could be implemented in any clinical microbiology laboratory worldwide without significant additional workload for the laboratory technicians. More generally, the method of the present invention also allows detecting any cellular enzyme capable of hydrolyzing a beta-lactam ring anti-microbial agent.

### SUMMARY

The present invention thus relates to a method for detecting, in a sample, a beta-lactamase activity, wherein said method is an impedance assay comprising the steps of:
(i) contacting the sample with at least one substrate of said beta-lactamase activity in at least one electrochemical cell; and
(ii) detecting an impedance variation in said electrochemical cell by collecting data points;
   wherein said at least one substrate comprises a beta-lactam ring.

In one embodiment, steps (i) and (ii) are performed simultaneously.

In one embodiment, said beta-lactamase activity is a carbapenemase activity or a cephalosporinase activity.

In one embodiment, the sample comprises a free enzyme. In another embodiment, the sample comprises a biological cell, preferably a bacteria. In one embodiment, said bacteria is a gram-negative bacteria selected from the group comprising enterobacterial cells and non-fermenting gram-negative bacteria cells.

In one embodiment, said substrate is selected from penams, cephems, monobactams, carbapenems, carbapenams, clavams, penems, carbacephems and oxacephems or a combination thereof, preferably said substrate is imipenem.

In one embodiment, the first step is performed in the presence of at least one cofactor salt, preferably ZnSO₄. In one embodiment, the first step is performed in the presence of at least one secondary salt, preferably CaCl₂, MnCl₂, MgCl₂, NaCl or KCl or any combination thereof such as, for example, CaCl₂ and MnCl₂ or CaCl₂ and MgCl₂.

In one embodiment, the method of the invention further comprises a step of lysing the biological cell. In another embodiment, said method does not comprise a step of lysing the biological cell.

Another object of the invention is a method for identifying a beta-lactamase activity, comprising the steps of:
(i) contacting a sample suspected to contain said beta-lactamase activity with at least one substrate thereof in at least one electrochemical cell, with at least one possible inhibitor of said beta-lactamase activity;
(ii) contacting the sample with said at least one substrate in at least one electrochemical cell, without the said at least one possible inhibitor;
(iii) detecting an impedance variation in said electrochemical cells of steps (i) and (ii) by collecting data points; and
(iv) comparing the impedance variations detected in step (iii);
   wherein said at least one substrate comprises a beta-lactam ring.

The present invention further relates to a method for screening candidate inhibitors for inhibiting a beta-lactamase activity, comprising the steps of:
(i) contacting a sample comprising said beta-lactamase activity with at least one substrate of said beta-lactamase activity and at least one candidate inhibitor, in at least one electrochemical cell;
(ii) contacting the sample with the said at least one substrate of said beta-lactamase activity without the said at least one candidate inhibitor;
(iii) detecting an impedance variation in said electrochemical cells of steps (i) and (ii) by collecting data points; and
(iv) comparing the impedance variations detected in step (iii);
   wherein said at least one substrate comprises a beta-lactam ring.

The present invention further relates to a method for screening candidate beta-lactam agents (preferably antimicrobial agents) that are not hydrolyzed by a beta-lactamase activity, comprising the steps of:
(i) contacting a sample comprising said beta-lactamase activity (either within a biological cell or in a free form) with at least one candidate beta-lactam agent, in at least one electrochemical cell;
(ii) contacting the sample with a known substrate of said beta-lactamase activity;
(iii) detecting an impedance variation in said electrochemical cells of steps (i) and (ii) by collecting data points; and
(iv) comparing the impedance variations detected in step (iii);
   wherein said at least one candidate anti-microbial agent comprises a beta-lactam ring.

Another object of the present invention is a system for detecting, in a sample, a beta-lactamase activity by measuring impedance of a working electrode, the system comprising:
a multiplexer comprising at least a 499 kΩ resistor and infinite resistor,
a working electrode made of an electro-conductive solid polymer transducer and coated with polyaniline;
an input to receive an input signal indicative of the potential to be applied between said working electrode and a reference electrode; and
an output to transmit an output signal indicative of the magnitude of the current flowing between a counter electrode and said working electrode;
said working and reference electrodes being adapted to be immerged into the sample or to be loaded with the sample;
a digital processor connected to a digital to analog converter for generating the input signal; and to an analog to digital converter for receiving at least one data point, which is a digital value;
a computer collecting at least 80 data points, preferably at least 400 data points, and calculating contiguous integrals of the data points in order to recover parameters summed to correspond to a global conductance.

In one embodiment, the polyaniline coated electrode is reusable.

In one embodiment, the working electrode is coated with polyaniline and at least one substrate of a beta-lactamase activity, preferably wherein said substrate is a carbapenem, more preferably imipenem.

In one embodiment, in the methods of the invention, the step of detecting an impedance variation comprises:
collecting exchanged charges in the form of data points in the electrochemical cell using a system as described hereinabove; and
calculating contiguous integrals of the data points and summing the integrals to obtain global conductance.

### DEFINITIONS

As used herein, the term “sensitivity” (also called the true positive rate) measures the proportion of actual positives which are correctly identified as such; while the term “specificity” (also called the true negative rate) refers to the proportion of negatives which are correctly identified as such

As used herein, the term “about” preceding a figure means plus or less 10% of the value of said figure.

Within the meaning of the invention, by “antimicrobial agent”, it is meant an agent that either kills or inhibits the growth of a microorganism, and more preferably of a bacteria.

By “beta-lactam ring”, it is meant a four-membered lactam, i.e. a four-membered cyclic amide having the general formula (I) below.

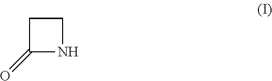

By “anti-microbial agent comprising a beta-lactam ring” or “beta-lactam anti-microbial agent”, it is meant an anti-microbial agent that contains a beta-lactam ring in its molecular structure. Beta-lactam anti-microbial agents in particular comprise penams (beta-lactam fused to thiazolidine rings), cephems (beta-lactam fused to 3,6-dihydro-2H-1,3-thiazine rings), monobactams (beta-lactam not fused to any other ring), carbapenems (beta-lactam fused to 2,3-dihydro-1H-pyrrole rings), carbapenams (beta-lactam fused to pyrrolidine rings), clavams (beta-lactam fused to oxazolidine), penems (beta-lactam fused to 2,3-dihydrothiazole rings), carbacephems (beta-lactam fused to 1,2,3,4-tetrahydropyrine rings) and oxacephems (beta-lactam fused to 3,6-dihydro-2H-1,3-oxazine rings). Penams in particular comprise penicillin, aminopenicillins (ampicillin, amoxicillin, bacampicillin and pivampicillin), carboxypenicillins (carbenicillin, ticarcillin, temocillin) and andureidopenicillins (azlocillin, mezlocillin, piperacillin). Cephems in particular comprise cephalosporins (such as, for example, cefotaxime), and cephamycins. Monobactams in particular comprise aztreonam, tigemonam, carumonam and nocardicin A. Carbapenems and penems in particular comprise ertapenem, imipenem, meropenem, doripenem, biapenem, panipenem, razupenem, tebipenem, lenapenem, tomopenem and faropenem.

As used herein, the term “beta-lactamase activity” refers to an enzyme activity capable of hydrolyzing an agent comprising a beta-lactam ring, such as, for example, a beta-lactam antimicrobial agent. According to the present invention, the term “beta-lactamase activity” thus includes carbapenemase activities (i.e. enzyme activities capable of hydrolyzing the beta-lactam structure of carbapenems antimicrobial agents) and cephalosporinase activities.

Within the meaning of the invention, by “electrochemical cell” it is meant a device allowing of either deriving electrical properties from chemical reactions or facilitating chemical reactions through the introduction of electrical energy. Electrochemical cells for use in the present invention are well known from the skilled person in the art.

Within the meaning of the invention, by “sensing material whose electronic properties are subject to variation” (either in response to the variation generated by the extemporaneous interaction between the at least one substrate and the enzyme, or when it interacts with an analyte), it is meant any material which is susceptible to a redox reaction, with consequent alteration of its electronic properties. By “alteration of its electronic properties”, it is meant any alteration resulting in a modification of the electrical charge of the said material, or any alteration resulting in a modification of the colour of said material.

### DETAILED DESCRIPTION

The present invention thus provides a method for detecting a beta-lactamase activity, wherein said method is based on the detection, by an impedance assay, of a variation generated by the extemporaneous interaction between an agent comprising a beta-lactam ring and a beta-lactamase activity. It is emphasized that the method of the present invention does not rely on the quantification of metabolites of a beta-lactam agent generated by the hydrolysis of said beta-lactam agent by said beta-lactamase activity.

The method of the invention indeed advantageously relies on the observation that the enzymatic hydrolysis of beta-lactam-containing substrates by beta-lactamases, which may be carbapenemases and/or by cephalosporinases, triggers a redox activity in the electrochemical cell, and optionally a pH variation. From this observation the Inventors carried out various experiences and implemented a method that happened to show an outstanding enhanced specificity and sensitivity as compared to the methods of the prior art. In particular, as demonstrated in the Examples, the method of the present invention, when used for identifying bacteria expressing an enzyme capable of hydrolyzing a beta-lactam ring, presents a specificity of more than 90%, preferably of more than 91, 92, 93, 94, 95, 96, 97, 98, 99% or even of 100%, combined with a sensitivity of more than 90%, preferably of more than 91, 92, 93, 94, 95, 96, 97% or more.

In one embodiment, the method of the invention allows identifying bacteria expressing a beta-lactamase activity using a bacterial suspension. In another embodiment, the method of the invention allows identifying bacteria expressing a beta-lactamase activity using only one colony recovered from a solid culture medium (such as, for example, a solid agar plate).

The present invention relates to a method for detecting, in a sample (preferably containing a biological cell), a beta-lactamase activity, wherein said method is an impedance assay comprising the steps of:
(i) contacting the sample suspected to contain said beta-lactamase activity with at least one substrate of said beta-lactamase activity in at least one electrochemical cell; and
(ii) detecting an impedance variation in said electrochemical cell by collecting data points;
   wherein said at least one substrate comprises a beta-lactam ring.

In one embodiment, the impedance variation is a conductimetric variation.

In one embodiment, the data points are digital values, preferably corresponding to exchanges charges.

In one embodiment, at least 80 data points, preferably at least 100, 200, 400, 600, 800 or 1000 data points or more are collected. In one embodiment, the method of the invention comprises calculating contiguous integrals of the data points in order to recover parameters which may be summed to correspond to a global conductance. In one embodiment, the method of the invention comprises calculating 1, 2, 3, 4, 5, 6, 7, 8, 9, 10 or more contiguous integrals.

The method of the invention is thus based on impedance, i.e. the method of the invention comprises measuring the impedance of an electrode. Indeed, as shown in the Examples, impedance assays of the invention allow identifying enzymatic activities, such as, for example, OXA-48 carbapenemase.

It is emphasized that the method of the invention does not include any cyclic voltametric assay.

In one embodiment, the interaction of the at least one substrate with the beta-lactamase activity generates a variation, which may be notably based on redox-activity. In one embodiment, the interaction of the at least one substrate with the beta-lactamase activity further generates a pH variation.

In one embodiment, the impedance variation is detected with monitoring means, wherein said monitoring means comprise one or more of a sensing material whose electronic properties are subject to variation generated by the extemporaneous interaction between the at least one substrate and the beta-lactamase activity and a detector arranged for monitoring electronic properties of the said sensing material.

In one embodiment, the at least one substrate generates at least one redox reaction when subjected to said enzyme activity, and the impedance variation is detected with monitoring means comprising one or more of a suitable sensing material.

In a particular embodiment of the invention, steps (i) and (ii) are performed subsequently.

However, in a preferred embodiment of the invention, steps (i) and (ii) are performed simultaneously, i.e. the detection step starts when contacting the substrate and the sample suspected to contain said beta-lactamase activity.

In one embodiment, the method of the invention is carried out at room temperature, such as, for example, at a temperature ranging from about 15 to about 25° C.

In a particular embodiment, the method of the present invention allows detecting at least one beta-lactamase, as referenced in the classification of Bush-Jacoby (functionnal groups 1 to 3, including subgroups; Bush K., “The ABCD's of β-lactamase nomenclature”, J Infect Chemother. 2013 Aug. 19(4):549-59). In a particular embodiment, the method of the present invention allows detecting at least one carbapenemase belonging to the Bush-Jacoby group 2df (molecular class D of Ambler), 2f (molecular class A), 3a and 3b (molecular class B1, B2 and B3). In a particular embodiment, the present method allows detecting at least one cephalosporinase.

Examples of enzymes that may be detected by the method of the present invention include, but are not limited to, CARB-type beta-lactamases (CARB-1 to 44), TEM-type with ESBL (extended-spectrum beta-lactamase) activity or without ESBL activity, resistant or not to inhibitor (TEM-1 to TEM-223), SHV-type ESBL or not ESBL, resistant or not to inhibitor (SHV-1 to SHV-193), CTX-M-type (CTX-M-1 to-170), ESBL enzymes such as PER enzymes (such as, for example, PER-1 to 8), VEB enzymes (such as, for example VEB-1 to 16), BEL enzymes (such as, for example BEL-1 to 3), OXA-type enzymes non ESBL, ESBL or carbapenemases (such as, for example OXA-1 to OXA-494) especially, OXA-48 and its carbapenemase variants (such as for example: OXA-162, 181, 204, 232, 244, 370, 494) GES-type enzymes including ESBL and carbapenemase (such as, for example, GES-1 to GES-27), Cephalosporinases enzymes such as CMY enzymes (such as for example CMY-1 to 135), DHA enzymes (such as, for example, DHA-1 to DHA-23), for example ACT-1 to 38, ACC-1 to 5, FOX-1 to 12, MIR-1 to 18, MOX-1 to 11, Extended-Spectrum AmpC (ESAC) enzymes, Carbapenemases such as for example KPC-type (wherein KPC stands for Klebsiella pneumoniae carbapenemase) enzymes (such as, for example, KPC-2 to 24), NDM-type (wherein NDM stands for New Delhi metallo-enzyme) enzymes (such as, for example, NDM-1 to NDM-16), VIM-type (wherein VIM stands for Verona integron-encoded metallo-beta-lactamase) enzymes (such as, for example, VIM-1 to VIM-46), IMP-type enzymes (such as, for example, IMP-1 to IMP-53), GIM-type enzymes (such as, for example, GIM-1 or GIM-2), IMI enzymes (such as, for example, IMI-1 to 9), IND-1 to 15, SFO enzymes, TLA enzymes, IBC enzymes, SME enzymes, NMC enzymes and CCRA enzymes.

Preferably, said enzyme is selected from CTX-M-type (CTX-M-1 to-170), OXA-type carbapenemases (such as, for example OXA-48-like, OXA-23, 24, 25, 26, 27, 40, 58, 72) especially, OXA-48 and its carbapenemase variants (such as for example: OXA-162, 181, 204, 232, 244, 370, 494) GES-type carbapenemase (such as, for example, GES-2 and 5), Carbapenemases such as for example KPC-type (wherein KPC stands for Klebsiella pneumoniae carbapenemase) enzymes (such as, for example, KPC-2 to 24), NDM-type (wherein NDM stands for New Delhi metallo-enzyme) enzymes (such as, for example, NDM-1 to NDM-16), VIM-type (wherein VIM stands for Verona integron-encoded metallo-beta-lactamase) enzymes (such as, for example, VIM-1 to VIM-46), IMP-type enzymes (such as, for example, IMP-1 to IMP-53), GIM-type enzymes (such as, for example, GIM-1 or GIM-2), IMI enzymes (such as, for example, IMI-1 to 9).

Preferably, said enzyme is selected from OXA-type carbapenemases (such as, for example, OXA-48, OXA-162, OXA-181, OXA-204 and OXA-232), KPC-type enzymes (such as, for example, KPC-2 or KPC-3), NDM-type enzymes (such as, for example, NDM-1 or NDM-5), VIM-type enzymes (such as, for example, VIM-1, VIM-2, VIM-4, VIM-27 and VIM-31), GIM-type enzymes (such as, for example, GIM-1), IMI enzymes (such as, for example, IMI-1 and IMI-2), and IMP-type enzymes (such as, for example, IMP-1, IMP-4, IMP-8, and IMP-11).

In one embodiment, the method of the invention allows detecting the presence of a beta-lactamase activity in a sample, and further allows identifying said beta-lactamase activity. Indeed, as shown in the Examples, and in particular in FIGS. 4, 5 and 7, the shape of the obtained curves varies according to the detected beta-lactamase activity. Therefore, according to one embodiment, the method of the invention allows detecting and identifying a specific beta-lactamase activity in a sample.

In one embodiment of the present invention, the sample to which the method of the invention is applied comprises at least one biological cell suspected to display at least one beta-lactamase activity, including a carbapenemase activity and/or a cephalosporinase activity.

In one embodiment of the present invention, the method of the invention is applied to a sample suspected to contain the beta-lactamase activity. In a particular embodiment, the said beta-lactamase activity results from the presence, in the said sample, of at least one beta-lactamase, such as, for example, at least one carbapenemase and/or at least one cephalosporinase.

In a particular embodiment, the at least one beta-lactamase enzyme is under a free form in the sample. In a particular embodiment, the at least one beta-lactamase enzyme is not purified. In a particular embodiment, the at least one beta-lactamase enzyme to be detected by the method of the invention is under a free form in the sample and is optionally purified by any suitable method known in the art.

In a particular embodiment, the method of the invention is thus implemented on a sample containing the free enzyme responsible for the enzyme activity, and present under a purified or non-purified form.

In another embodiment, the beta-lactamase activity is, for instance, present inside a biological cell, or in the intermembrane space thereof, such as in the periplasm of gram-negative bacteria. In another embodiment, the beta-lactamase activity is displayed on the outside membrane and/or envelope of a biological cell. In a further embodiment, the beta-lactamase activity is displayed both inside and outside a biological cell.

Within the meaning of the invention, by “biological cell”, it is meant a biological unit enclosed with a membrane. In a particular embodiment of the invention, the biological cell to which the method of the invention is applied is a bacteria, preferably a gram-negative or gram-positive bacteria. In a particular embodiment, the biological cell to which the method of the invention is applied is a gram-negative bacteria. In a particular embodiment, the biological cell to which the method of the invention is applied is a gram-positive bacteria. In a particular embodiment of the invention, the biological cell to which the method of the invention is applied is a gram-negative bacteria selected from the group comprising enterobacterial cells (Enterobacteriaceae) and non-fermenting gram-negative bacteria cells (such as for instance Acinetobacter spp and Pseudomonas spp). In a particular embodiment of the invention, the biological cell to which the method of the invention is applied is a bacteria selected from the group comprising Acinetobacter spp including baumannii, pittii, hemolitycus and junii, Aeromonas caviae, Citrobacter amalonaticus, Citrobacter braakii, Citrobacter freundii, Citrobacter youngae, Enterobacter aerogenes, Enterobacter asburiae, Enterobacter cloacae, Escherichia coli, Klebsiella oxytoca, Klebsiella pneumoniae, Morganella morganii, Proteus mirabilis, Proteus rettgeri, Proteus vulgaris, Providencia stuartii, Providencia vermicola, Pseudomonas spp. including aeruginosa and putida, Salmonella enterica, Serratia marcescens and Shigella flexneri.

In a particular embodiment, the method of the present invention is used for detecting carbapenemase-producing bacteria including Enterobacteriaceae and gram-negative non-fermenting bacteria.

In one embodiment, the biological cell was previously recovered and isolated from a biologic sample of an individual (such as, for example, urine sample, saliva sample, respiratory samples (such as for example bronchoalveolar lavage, endotracheal aspirate, nasopharyngal aspirate and the like), wounds sample, skin sample and soft tissue sample, stool sample, screening samples (rectal, perineal, or skin swabs) or blood sample). In another embodiment, the biological cell was previously recovered by sampling, such as, for example, from environmental sampling or sampling on alimentary products.

In one embodiment, the biological cells recovered from a biological sample or from any other sampling is first treated for increasing bacterial concentration, such as, for example, for obtaining a concentration of biological cells of at least about 10⁷ cells/mL, preferably at least 10⁸, more preferably at least 10⁹ cells/mL, even more preferably at least about 10¹⁰ cells/mL and still even more preferably at least about 10¹¹ cells/mL or more.

In one embodiment, the biological cells recovered from a biological sample or from any other sampling is first treated for increasing the number of bacterial cells, such as, for example, for obtaining a number of biological cells of at least about 10³ cells, preferably at least about 10⁴ cells, more preferably at least about 10⁵ cells and even more preferably at least 10⁶ cells or more.

Examples of treatments include, but are not limited to, culture of bacterial cells, centrifugation, filtration and the like.

In one embodiment, the biological cell of the invention was previously grown in a culture medium before implementing the method of the present invention, such as, for example, a liquid or solid culture medium, preferably on a solid culture medium. Examples of solid culture medium that may be used include, but are not limited to, Trypticase Soy Agar with 5% Sheep Blood (TSA II) (Becton Dickinson), Brilliance CRE Agar (Oxoid), Chocolate agar PolyViteX (BioMérieux), ChromID Carba (BioMérieux), ChromID OXA-48 (BioMérieux), Columbia Agar with 5% Sheep Blood (Becton Dickinson), Columbia Agar With 5% Horse Blood (Becton Dickinson), chromID® CPS (BioMérieux), Drigalski Lactose Agar (BioRad), chromID® ESBL (BioMérieux), KPC CHROMagar (Biotrading), Mac Conkey agar (BioMérieux), Mueller Hinton II Agar (Becton Dickinson), Mueller Hinton Agar (powder, Oxoid) with or without ZnSO₄ (at a concentration of 35, 70 or 140 μg/ml), BBL Chromagar Orientation (Becton Dickinson), Nutrient Broth+agar (Oxoid), or UriSelect™ 4 Medium (BioRad). Preferably, the culture medium is selected from Trypticase Soy Agar with 5% Sheep Blood (TSA II) (Becton Dickinson), Brilliance CRE Agar (Oxoid), Chocolate agar PolyViteX (BioMérieux), ChromID Carba (BioMérieux), ChromID OXA-48 (BioMérieux), Columbia Agar with 5% Sheep Blood (Becton Dickinson), Columbia Agar With 5% Horse Blood (Becton Dickinson), Drigalski Lactose Agar (BioRad), chromID® ESBL (BioMérieux), KPC CHROMagar (Biotrading), Mueller Hinton Agar (powder, Oxoid) with or without ZnSO₄ (at a concentration of 35, 70 or 140 μg/ml), BBL Chromagar Orientation (Becton Dickinson), and Nutrient Broth+agar (Oxoid).

In one embodiment, the biological cell is grown in culture for about 18 to 24 hours at 37° C. before implementing the method of the invention. In another embodiment, the biological cell is stored at room temperature (i.e. at a temperature ranging from about 15° C. to about 25° C.) for at most 48 hours, preferably for at most 24 hours.

In one embodiment, the biological cell was thus previously recovered from a solid culture medium.

Within the meaning of the invention, by “substrate of said beta-lactamase activity” or “substrate”, it is meant any compound suitable to be hydrolyzed by a beta-lactamase activity. In a particular embodiment, the substrate for use in the method of the invention contains a beta-lactam ring.

In a particular embodiment, the substrate for use in the invention is an anti-microbial agent, preferably a beta-lactam anti-microbial agent. In a particular embodiment, the substrate for use in the invention is selected from the group consisting of penams, cephems, monobactams, carbapenems, carbapenams, clavams, penems, carbacephems and oxacephems or a combination thereof. In a particular embodiment, the substrate for use in the invention is selected from the group consisting of penicillin, aminopenicillins (ampicillin, amoxicillin, bacampicillin and pivampicillin), carboxypenicillins (carbenicillin, ticarcillin, temocillin), andureidopenicillins (azlocillin, mezlocillin, piperacillin), cephalosporins, cephamycins, aztreonam, tigemonam, carumonam, nocardicin A, ertapenem, imipenem, meropenem, doripenem, biapenem, panipenem, razupenem, tebipenem, lenapenem, tomopenem and faropenem, or a combination thereof. In a particular embodiment, the substrate for use in the method of the invention is a carbapenem, preferably selected from the group consisting of ertapenem, meropenem, doripenem, biapenem and imipenem, or a combination thereof. In a particular embodiment, the substrate for use in the invention is imipenem, temocillin or cefotaxime, preferably imipenem.

In a particular embodiment of the invention, contacting the at least one substrate and the said enzyme activity generates a reduction or oxidation of the electrode. In a particular embodiment of the invention, contacting the at least one substrate and the said enzyme activity generates an acidification or basification of the electrode. In a particular embodiment of the invention, contacting the at least one substrate and the said enzyme activity generates a reduction and acidification of the electrode. In a particular embodiment of the invention, contacting the at least one substrate and the said enzyme activity generates a reduction and basification of the electrode. In a particular embodiment of the invention, contacting the at least one substrate and the said enzyme activity generates an oxidation and basification of the electrode. In a particular embodiment of the invention, contacting the at least one substrate and the said enzyme activity generates an oxidation and acidification of the electrode.

In one embodiment, the substrate, preferably imipenem, is used in a concentration ranging from about 0.1 to about 20 mg/mL, more preferably from about 1 to about 10 mg/mL, even more preferably of about 3 to 6 mg/mL, and still even more preferably about 6 mg/mL

In a particular embodiment of the invention, the step of contacting the sample with a substrate of the beta-lactamase activity is performed in the presence of at least one cofactor salt. According to the invention, by “at least one cofactor salt”, it is meant any salt or combination thereof, the presence of which is required for or enhances the beta-lactamase activity to be detected or monitored. In a particular embodiment, no cofactor salt is added for contacting the sample with the substrate of the beta-lactamase activity. In another embodiment, the cofactor salt is selected from the group consisting of transition metal and post-transition metal salts or combinations thereof. Within the invention, by “transition metal salts” is meant a salt of a metal comprised in the group consisting in: Sc, Ti, V, Cr, Mn, Fe, Co, Ni, Cu, Zn, Y, Zr, Nb, Mo, Tc, Ru, Rh, Pd, Ag, Cd, La, Hf, Ta, W, Re, Os, Ir, Pt, Au, Hg, Ac, Unq, Unp, Unh, Uns, Uno, Une, and Unr, or combinations thereof. Within the invention, by “post-transition metal salts” is meant a salt of a metal comprised in the group consisting of Al, In, Sn, Bi, Pb, Ga, Ge, Sb, Po, Uut, Uuq, Uup, Uuh and Tl, or combinations thereof.

In a particular embodiment of the invention, the cofactor salt for use in the method of the invention is ZnSO₄. In a particular embodiment, the cofactor salt for use in the method of the invention is present in an amount of greater than 0 mM to about 1 M. In a particular embodiment, the cofactor salt for use in the method of the invention is present in an amount of greater than 0 mM to about 25 mM. In a particular embodiment, the cofactor salt for use in the method of the invention is present in an amount of from about 0.01 mM to about 0.15 M. In one embodiment, the cofactor salt is present in an amount ranging from about 0.01 mM to about 1.75 mM, more preferably from about 0.05 mM to about 1 mM, more preferably in an amount of about 0.075 to about 0.1 mM, and even more preferably of about 0.077 or about 0.1 mM. In another embodiment, the cofactor salt is present in an amount ranging from about 0.1 mM to about 0.5 mM, preferably of about 0.3 mM.

In one embodiment, the sample comprising the enzymatic activity further comprises from about 0.01 mM to about 1.75 mM, preferably from about 0.075 to about 1 mM, more preferably about 0.077 or about 0.1 mM of ZnSO₄. In another embodiment, the sample comprising the enzymatic activity further comprises from about 0.1 mM to about 0.5 mM, preferably of about 0.3 mM of ZnSO₄.

In another embodiment, the substrate is in a medium comprising from about 0.01 mM to about 1.75 mM, preferably from about 0.075 to about 1 mM, more preferably about 0.077 or about 0.1 mM of ZnSO₄. In another embodiment, the substrate is in a medium comprising from about 00.1 mM to about 0.5 mM, preferably of about 0.3 mM of ZnSO₄.

In a particular embodiment of the invention, the step of contacting the sample with a substrate of the beta-lactamase activity is performed in the presence of at least one secondary salt selected from the group consisting in alkali metal salts (group IA) and alkaline earth salts (group IIA).

According to the invention, by “alkali metal salts”, it is meant a salt of a metal comprised in the group consisting in: Li, Na, K, Rb, Cs, and Fr, or any combination thereof.

According to the invention, by “alkali earth metal salts”, it is meant a salt of a metal comprised in the group consisting in: Be, Mg, Ca, Sr, Ba and Ra, or any combination thereof.

In a particular embodiment, no secondary salt is added for contacting the sample with the substrate of the beta-lactamase activity.

In another embodiment, the secondary salt for use in the method of the invention is selected from the group consisting of CaCl₂, MgCl₂, MnCl₂, MgSO₄, NH₄Cl, NaCl, KCl, CaSO₄, ZnCl₂ or a combination thereof. In a particular embodiment, the secondary salt for use in the method of the invention is a combination of CaCl₂ and MgCl₂.

In a particular embodiment, the cofactor salt for use in the method of the invention is present in an amount of from greater than 0 M to 1 M. In a particular embodiment, the cofactor salt for use in the method of the invention is present in an amount of from greater than 50 mM to 300 mM, such as, for example, about 100 mM, 150 mM or 200 mM. In another embodiment, the cofactor salt for use in the method of the invention is present in an amount ranging from about 15 to about 50 mM, preferably of about 17 mM or of about 34 mM.

In one embodiment, the secondary salt is CaCl₂ in a concentration of about 100, 150 or 200 mM, preferably of about 150 mM. In another embodiment, the secondary salt is MnCl₂ in a concentration of about 100, 150 or 200 mM, preferably of about 150 mM.

In another embodiment, the secondary salt is CaCl₂ in a concentration of about 17 or 34 mM. In another embodiment, the secondary salt is MnCl₂ in a concentration of about 17 or 34 mM.

In another embodiment, the secondary salt is a combination of CaCl₂ and MgCl₂, wherein preferably CaCl₂ is in a concentration of about 50, 75 or 100 mM (preferably of about 75 mM) and MgCl₂ is in a concentration of about 50, 75 or 100 mM (preferably of about 75 mM).

In another embodiment, the secondary salt is a combination of CaCl₂ and MgCl₂, wherein preferably CaCl₂ is in a concentration of about 10 to 30 Mm, preferably 17 mM and MgCl₂ is in a concentration of about 10 to 30 Mm, preferably 17 mM.

In another embodiment, the secondary salt is a combination of CaCl₂ and MnCl₂, wherein preferably CaCl₂ is in a concentration of about 50, 75 or 100 mM (preferably of about 75 mM) and MnCl₂ is in a concentration of about 50, 75 or 100 mM (preferably of about 75 mM).

In another embodiment, the secondary salt is a combination of CaCl₂ and MnCl₂, wherein preferably CaCl₂ is in a concentration of about 10 to 30 Mm, preferably 17 mM and MnCl₂ is in a concentration of about 10 to 30 Mm, preferably 17 mM.

In one embodiment, the secondary salt is NaCl or KCl. Preferably, NaCl or KCl are present in a concentration of about 150 mM to about 5 M. In one embodiment, NaCl is in a concentration of about 5 M or of about 4M. In another embodiment, NaCl is in a concentration of about 1.2 M. In another embodiment, KCl is in a concentration of about 3 M or of about 4 M.

The present invention further relates to a buffer comprising at least one cofactor salt, preferably ZnSO₄. Preferably, the cofactor salt is in a concentration of about 0.01 mM to about 1.75 mM, preferably from about 0.075 to about 1 mM, more preferably about 0.077 or about 0.1 mM; or in a concentration ranging from about 0.1 mM to about 0.5 mM, preferably of about 0.3 mM.

In one embodiment, the buffer further comprises a substrate, preferably imipenem. Preferably, the substrate is in a concentration ranging from about 0.1 to about 20 mg/mL, more preferably from about 1 to about 10 mg/mL, even more preferably of about 3 to 6 mg/mL, and still even more preferably about 6 mg/mL.

The present invention further relates to a buffer comprising at least one secondary salt.

In one embodiment, the secondary salt is CaCl₂ or MnCl₂ in a concentration of about 100, 150 or 200 mM, preferably of about 150 mM. In one embodiment, the secondary salt is CaCl₂ or MnCl₂ in a concentration ranging from about 10 to about 50 mM, preferably of about 17 mM or of about 34 mM. In another embodiment, the secondary salt is a combination of CaCl₂ and MgCl₂, wherein preferably CaCl₂ is in a concentration of about 50, 75 or 100 mM (preferably of about 75 mM) and MgCl₂ is in a concentration of about 50, 75 or 100 mM (preferably of about 75 mM). In another embodiment, the secondary salt is a combination of CaCl₂ and MgCl₂, wherein preferably CaCl₂ is in a concentration ranging from about 10 to about 30 mM (preferably of about 17 mM) and MgCl₂ is in a concentration ranging from about 10 to about 30 mM (preferably of about 17 mM). In another embodiment, the secondary salt is a combination of CaCl₂ and MnCl₂, wherein preferably CaCl₂ is in a concentration of about 50, 75 or 100 mM (preferably of about 75 mM) and MnCl₂ is in a concentration of about 50, 75 or 100 mM (preferably of about 75 mM). In another embodiment, the secondary salt is a combination of CaCl₂ and MnCl₂, wherein preferably CaCl₂ is in a concentration ranging from about 10 to about 30 mM (preferably of about 17 mM) and MnCl₂ is in a concentration ranging from about 10 to about 30 mM (preferably of about 17 mM). In one embodiment, the secondary salt is NaCl or KCl. Preferably, NaCl or KCl are present in a concentration of about 150 mM to about 5 M. In one embodiment, NaCl is in a concentration of about 5 M or 4 M. In another embodiment, NaCl is in a concentration of about 1.2 M. In another embodiment, KCl is in a concentration of about 3 M or of about 4 M.

The present invention further relates to a buffer comprising at least one cofactor salt and at least one secondary salt. In one embodiment, the buffer of the invention comprises ZnSO₄ (preferably in a concentration ranging from about 0.05 to about 0.1 mM, more preferably of about 0.077 mM or ranging from about 0.1 mM to about 0.5 mM, preferably of about 0.3 mM), CaCl₂ (preferably in a concentration ranging from about 5 to about 50 mM, more preferably from about 15 to about 20 mM, and even more preferably of about 17 mM) and MgCl₂ (preferably in a concentration ranging from about 5 to about 50 mM, more preferably from about 15 to about 20 mM, and even more preferably of about 17 mM). In another embodiment, the buffer of the invention comprises ZnSO₄ (preferably in a concentration ranging from about 0.05 to about 0.1 mM, more preferably of about 0.077 mM from about 0.1 mM to about 0.5 mM, preferably of about 0.3 mM), and NaCl (preferably in a concentration ranging from about 0.1 M to about 5 M, more preferably from about 0.5 M to about 2 M, and even more preferably of about 1.2 M or in a concentration of about 4 M).

In one embodiment, the buffer further comprises a substrate, preferably imipenem. Preferably, the substrate is in a concentration ranging from about 0.1 to about 20 mg/mL, more preferably from about 1 to about 10 mg/mL, even more preferably of about 3 to 6 mg/mL, and still even more preferably about 6 mg/mL.

In a particular embodiment, the method of the invention further comprises a step of lysing the biological cell. In a particular embodiment, the step of lysing the biological cell is performed during or before step (i). In such an embodiment, the said biological cell is lysed by any chemical and/or physical means known in the art. In a particular embodiment, the lysis of the biological cell is performed by subjecting the sample containing the biological cell to a solvent and/or detergent treatment, and/or by vigorous shaking of the medium containing the said biological cell.

In another particular embodiment, the method of the invention does not comprise any step of lysing the biological cell. The inventors have indeed surprisingly discovered that the biological cell may be directly contacted with the substrate of the enzyme activity, absent any lysis of the biological cell. In a particular embodiment, the detection of the enzyme activity in absence of cell lysis is enhanced in the presence of a secondary salt as described above.

In a particular embodiment, the electrochemical cell for use in the present invention comprises at least two electrodes, i.e. at least a working electrode and a counter electrode. In a particular embodiment, the electrochemical cell for use in the present invention comprises three electrodes, including a working electrode, a counter electrode and a reference electrode.

In a particular embodiment, the sensing material for use in the method of the invention comprises a polymer susceptible to a redox reaction with consequent alteration of its electronic properties.

In a particular embodiment, the sensing material for use in the invention is selected from the group consisting of polyanilines, polythiophenes, and polypyrroles, and combinations thereof.

In a particular embodiment, the sensing material for use in the invention is polyaniline (also referred to as “PANI”). Polyaniline can act as a mediator for both pH-metry and redox titration. Polyaniline is a very convenient polymeric material when used as a solid electrochemical transducer owing to its many interesting intrinsic combinations of redox and acid-base states. FIG. 1 displays the redox equilibriums of polyaniline in vertical and acid-base equilibriums thereof in horizontal. The average molecular structure of this polymer has been determined by Mac-Diarmid et al. (MacDiarmid et al., Synth. Meth. 18(1987), 285-290; Chiang et MacDiarmid, Synth. Met., 13 (1986), 193-205), which also have described the possible conversions between PANI redox and acid-base forms.

In an embodiment of the present invention, a sensing material, preferably polyaniline, is displayed on an electrode and can be used to detect oxidation or reduction events together with acidification or basification. To do so, the initial redox and acido-basic states of the sensing material are carefully chosen in order to maximize its electrochemical response during detection. In a particular embodiment, the initial state of polyaniline for use in the present invention is preferably chosen according to conductivity, redox potential and pH phase diagrams described in Focke et al. (J. Phys. Chem., 1987, 91: 5813-5818).

In one embodiment, the electrode for use in the method of the invention is reusable. Indeed, the Inventors have proven (see Examples) that the electrode may be used up to at least 10 times, preferably 15 times, more preferably 20 times without any troubles, thanks to a procedure erasing former results obtained with the electrode.

Before use, freshly electrosynthesized polyaniline electrodes are in a partially oxidized state which is non-conductive. Starting from this state, any redox reaction giving electrons (reduction) will reduce polyaniline and hence raise its conductivity. A pH decrease will also raise conduction but with a magnitude depending on the polyaniline's redox state.

In one embodiment, the method of the invention comprises a first step constituting a “RESET step”. This step puts all electrodes in the same initial state just before their use. Thanks to this step, it is possible to reuse one electrode several times. In one embodiment, during said RESET step, a potential, preferably ranging from about 500-1000 mV, more preferably of about 800 mV is applied on the electrodes for a certain period of time, preferably for 5-60 seconds, more preferably 15 seconds. In another embodiment, the RESET step consists in plunging the electrodes for a certain period of time in a medium containing an oxidant reagent (such as, for example, Ammonium persulfate (NH₄)₂S₂O₈).

The present invention further relates to an electrode regeneration or cleaning buffer.

Within the meaning of the invention, by “detector”, it is meant any device allowing an electronic variation of the sensing material to be detected. In a particular embodiment of the invention, the detection of the electronic variation is performed by electrical measurement between electrodes. In such an embodiment, the detector for use in the method of the invention may comprise any type of conductimeter.

In a particular embodiment, the detector is operationally coupled to an electrode covered with or comprising a sensing material as defined above, and preferably polyaniline.

In a particular embodiment of the invention, the substrate is on or within the said sensing material. In a particular embodiment, the substrate is coupled to the electrochemically active sensing material by any covalent or non-covalent coupling method known in the art.

In an embodiment of the invention, the substrate is chemically coupled to the electrochemically active sensing material covering at least one electrode, or elsewhere on the electrode (supporting material, counter electrode, etc). In another embodiment, the substrate is lyophilised on the electrochemically active sensing material or elsewhere on the electrode (supporting material, counter electrode, etc). In another embodiment, the substrate is incorporated in the electrochemically active sensing material or into an inert soluble or insoluble material placed on the electrode (e.g. supporting material, counter electrode, etc).

The present invention further concerns an electrode coated with a sensing material whose electronic properties are (i) subject to redox variation and optionally to acid-base variation or (ii) subject to variation generated by the extemporaneous interaction between the at least one substrate and the beta-lactamase activity. The sensing material for use in the present invention is as defined above and is more specifically polyaniline. In a particular embodiment, the electrode of the invention is further coated with the at least one substrate for detecting the enzyme activity according to the invention, and is preferably coated with a beta-lactam anti-microbial agent, preferably a carbapenem, more preferably with imipenem.

In a particular embodiment, the polyaniline is in an emeraldine state or in a more oxidized state than emeraldine or in any other state.

The present invention further concerns the use of an electrode as defined above in a method for detecting a beta-lactamase activity in a sample.

The present invention further relates to a system that may be used in the methods of the present invention, wherein said system comprises:
a multiplexer comprising at least a 499 kΩ resistor and infinite resistor,
a working electrode made of an electro-conductive solid polymer transducer and coated with polyaniline;
an input to receive an input signal indicative of the potential to be applied between said working electrode and a reference electrode; and
an output to transmit an output signal indicative of the magnitude of the current flowing between a counter electrode and said working electrode;
said working and reference electrodes being adapted to be immerged into the sample or to be loaded with the sample;
a digital processor connected to a digital to analog converter for generating the input signal; and to an analog to digital converter for receiving at least one data point, which is a digital value;
a computer collecting data points, and calculating contiguous integrals of the data points in order to recover parameters summed to correspond to a global conductance.

In one embodiment, at least 80 data points, preferably at least 100, 200, 400, 600, 800 or 1000 data points or more are collected. In one embodiment, 1, 2, 3, 4, 5, 6, 7, 8, 9, 10 or more contiguous integrals are calculated.

In one embodiment, the polyaniline coated electrode is reusable.

In one embodiment, the working electrode is coated with polyaniline and at least one substrate of a beta-lactamase activity, preferably wherein said substrate is a carbapenem, more preferably imipenem.

In one embodiment, the system of the invention further comprises a capacitor in parallel to the 499 kΩ resistor.

Within the meaning of the invention, by “sample”, it is meant at least one biological cell suspected to display a beta-lactamase activity or a solution comprising at least one enzyme responsible for this activity.

In one embodiment, the first step of the method of the invention thus comprises contacting:
a sample as defined above, with
a substrate;
   optionally in presence of at least one secondary salt and/or at least one cofactor salt as described hereinabove.

In an embodiment of the invention, the sample corresponds to a medium containing the biological cell or the free enzyme, wherein the at least one substrate is further added.

In another embodiment, the at least one substrate is contained in a medium wherein the biological cell or the free enzyme are further added.

By “medium”, it is meant any suitable environment for implementing the method of the invention: the medium may thus, for instance, be liquid or semi-solid, such as a gel. In another embodiment of the invention, the medium is water, preferably demineralized water or water for injection, or a saline buffer. In another embodiment, the biological cell or the free enzymatic activity is contacted with the at least one substrate in absence of a medium.

In one embodiment, whole or part of the monitoring means comprising the one or more sensing material, e.g. an electrode covered with polyaniline, is plunged in a medium containing the sample to be tested and, optionally further comprising at least one substrate and/or at least one cofactor salt and/or at least one secondary salt. Preferably, according to this embodiment, the monitoring means is in a vertical position.

In one embodiment, a first medium is prepared comprising the biological cell or the enzyme to be tested, and optionally at least one cofactor and/or at least one secondary salt, and the substrate is sub-sequentially added within the first medium. Preferably, the substrate is added concomitantly with the plunge of the monitoring means within the first medium.

In another embodiment,
a first medium is prepared comprising the biological cell or the enzyme to be tested, and optionally at least one secondary salt;
a second medium is prepared comprising the substrate and optionally at least one cofactor salt, and
the first and second media are mixed in a third medium, wherein the monitoring means are plunged.

Preferably, the mixing step is concomitant with the plunge of the monitoring means within the third medium.

In another embodiment, whole or part of the monitoring means comprising the one or more sensing material, e.g. an electrode covered with polyaniline, and the substrate on or within the sensing material, is plunged in a medium containing the biological cell or the enzyme to be tested, and optionally further comprising at least one cofactor salt and/or at least one secondary salt.

In a particular embodiment, the method of the invention is performed by loading the sample, and optionally said at least one substrate, onto the sensing material.

In one embodiment, the sample is in a liquid form and a drop of said sample is loaded onto the said sensing material. Preferably, according to this embodiment, the monitoring means is in a horizontal position.

According to a first embodiment, the method of the invention comprises:
preparing a first liquid medium comprising the biological cell or the free enzyme to be tested, and optionally at least one secondary salt;
preparing a second liquid medium comprising a substrate, and optionally at least one cofactor salt;
mixing the first and second media, thereby obtaining a third medium; and
loading a drop of the third medium onto the sensing material.

Preferably, the drop has a volume ranging from about 30 to about 60 μL, preferably a volume of about 50 μL.

According to a second embodiment, the method of the invention comprises:
preparing a liquid medium comprising a substrate, and optionally at least one cofactor salt;
loading a drop of the liquid medium onto the sensing material;
preparing a suspension comprising the biological cell or the free enzyme and optionally at least one secondary salt; and
loading a drop of the suspension onto the drop of liquid medium.

In one embodiment, the drop of liquid medium has a volume ranging from about 1 μL to about 50 μL, preferably from about 2 to about 10 μL, and more preferably of about 5 μL.

In another embodiment, the drop of suspension has a volume ranging from about 1 μL to about 50 μL, preferably from about 35 to about 50 μL, and more preferably of about 45 μL.

In another embodiment, the sum of the volumes of the drop of liquid medium and of the drop of suspension ranges from about 30 to about 60 μL, preferably is of about 50 μL.

According to a third embodiment, the method of the invention comprises:
preparing a buffer comprising a substrate, and optionally at least one cofactor salt and optionally at least one secondary salt;
preparing a suspension of the biological cell or the free enzyme within said buffer obtained in the previous step; and
loading a drop of said suspension onto the sensing material.

Preferably, the drop has a volume ranging from about 30 to about 60 μL, preferably a volume of about 50 μL.

Preferably, the buffer comprises ZnSO₄ (preferably in a concentration ranging from about 0.05 to about 0.1 mM, more preferably of about 0.077 mM), CaCl₂ (preferably in a concentration ranging from about 5 to about 50 mM, more preferably from about 15 to about 20 mM, and even more preferably of about 17 mM) and MgCl₂ (preferably in a concentration ranging from about 5 to about 50 mM, more preferably from about 15 to about 20 mM, and even more preferably of about 17 mM).

According to a fourth embodiment, the method of the invention comprises:
loading a colony of the biological cells onto the sensing material;
preparing a buffer comprising a substrate, and optionally at least one cofactor salt and optionally at least one secondary salt; and
loading a drop of said buffer onto the biological cells previously loaded onto the sensing material.

Preferably, the drop has a volume ranging from about 30 to about 60 μL, preferably a volume of about 50 μL.

In one embodiment, the buffer comprises NaCl (preferably in a concentration ranging from about 0.1 M to about 5 M, more preferably from about 0.5 M to about 2 M, and even more preferably of about 1.2 M) and ZnSO₄ (preferably in a concentration ranging from about 0.05 to about 0.1 mM, more preferably of about 0.077 mM). In another embodiment, the buffer comprises 4 M NaCl and 0.3 mM ZnSO₄.

In a particular embodiment, when said at least one substrate is on or within the said sensing material, the method of the invention is performed by loading the sample onto the said sensing material. In a particular embodiment, the sample optionally comprising the at least one substrate, is mixed with at least one cofactor salt and/or at least one secondary salt before being loaded, or when loaded onto the said sensing material.

The present invention further concerns a method for identifying a beta-lactamase activity, comprising the steps of:
(i) contacting a sample suspected to contain said beta-lactamase activity (either within a biological activity or in a free form) with at least one substrate thereof in at least one electrochemical cell, with at least one possible inhibitor of said beta-lactamase activity;
(ii) contacting the sample with said at least one substrate thereof in at least one electrochemical cell, without the said at least one possible inhibitor;
(iii) detecting a impedance variation in said electrochemical cells of steps (i) and (ii) by collecting data points; and
(iv) comparing the impedance variations detected in step (iii);
   wherein said at least one substrate comprises a beta-lactam ring.

In one embodiment, the interaction of the at least one substrate with the enzyme activity generates an impedance variation based in particular on redox-activity. In another embodiment, said at least one substrate generates at least one redox-reaction when subjected to said enzyme activity.

In one embodiment, the impedance variation is detected with monitoring means as described hereinabove.

In the method of identifying a beta-lactamase activity according to the present invention, the inhibitory effect of several specific inhibitors known for their capacity to prevent the hydrolysis of beta-lactam rings anti-microbial agents by various enzyme activities may be tested subsequently or simultaneously. Any inhibitor known in the art for preventing the hydrolysis of beta-lactam rings may advantageously be used in the identification method of the invention. As regards more specifically to the identification of carbapenemases, suitable inhibitors for use in the method of the present invention comprise, but are not limited to:
Class A carbapenemases inhibitors (e.g. clavulanic acid salts, preferably in a concentration of from 0.1 mg/L to 10 mg/L, preferably at a concentration of 2 mg/L; tazobactam, preferably in a concentration of from 0.1 mg/L to 10 mg/L, more preferably at a concentration of 4 mg/L; sulbactam, preferably in a concentration of from 0.1 mg/L to 10 mg/L, more preferably at a concentration of 4 mg/L; boronic acid salts and derivatives thereof (for KPC carbapenemases only), preferably in a concentration of from 10 to 10000 mg/L);
Class B carbapenemases inhibitors (e.g. cation chelators such as, for instance, EDTA, preferably in a concentration of from 0.1 to 10 mM, more preferably at a concentration of 10 mM; or dipicolonic acid, preferably in a concentration of from 10 to 10000 mg/L);
Class C carbapenemase inhibitors (e.g. boronic acid salts and derivatives thereof, preferably in a concentration of from 10 to 10000 mg/L; Oxacillin and cloxacillin, preferably in a concentration of from 100 to 8000 mg/L, more preferably in a concentration of 4000 mg/L); and
Class D carbapenemases inhibitors (e.g. avibactam, preferably in a concentration of from 0.1 to 10 mg/L, more preferably at a concentration of 4 mg/L).

In a particular embodiment, the method for identifying a beta-lactamase activity of the present invention allows determining the classification of said enzyme activity among the known classes of beta-lactamases, including carbapenemases and/or cephalosporinases.

When an impedance variation is comparably detected in the presence and in absence of a possible inhibitor, it should then be inferred that the tested inhibitor is not preventing the hydrolysis of beta-lactam antimicrobial-agents and therefore that the tested enzyme activity does not belong to the class of beta-lactamase which is targeted by the tested inhibitor. Similarly, if an impedance variation is detected in absence of the tested inhibitor but is no more detected in presence of this inhibitor, it should then be inferred that the substrate is no more hydrolyzed by the enzyme activity and that the tested enzyme activity thus belongs to the specific class of enzymes usually targeted by the inhibitor. The corresponding class of enzyme may further be deduced from one or more comparison tests performed with one or more known inhibitors possessing distinct specificities.

A further object of the present invention concerns a method for screening candidate inhibitors for inhibiting a beta-lactamase activity, comprising the steps of:
(i) contacting a sample comprising said beta-lactamase activity (either within a biological cell or in a free form) with at least one substrate of said enzyme activity and at least one candidate inhibitor, in at least one electrochemical cell;
(ii) contacting the sample with said at least one substrate of said beta-lactamase activity without the said at least one candidate inhibitor;
(iii) detecting an impedance variation in said electrochemical cells of steps (i) and (ii) by collecting data points; and
(iv) comparing the impedance variations detected in step (iii);
   wherein said at least one substrate comprises a beta-lactam ring.

In one embodiment, the interaction of the at least one substrate with the enzyme activity generates an impedance variation in particular based on redox-activity. In another embodiment, said at least one substrate generates at least one redox-reaction when subjected to said enzyme activity.

In one embodiment, the impedance variation is detected with monitoring means as described hereinabove.

In one embodiment, the screening method of the invention aims at identifying new inhibitors capable of preventing the hydrolysis of beta-lactam anti-microbial agents by a specific enzyme activity. In the method of the invention, when an impedance variation is comparably detected in the presence and in absence of a candidate inhibitor, it should then be inferred that the tested candidate inhibitor is not able of preventing the hydrolysis of beta-lactam antimicrobial-agents by the enzyme activity contained in the tested sample, and therefore that it could not be considered as an actual inhibitor of the tested class of enzyme. On the contrary, if an impedance variation is detected in absence of the tested candidate inhibitor but is no more detected in presence of this candidate inhibitor, it should then be inferred that the tested candidate inhibitor is capable of inhibiting the tested class of enzymes hydrolyzing beta-lactam ring antimicrobial agents. A same candidate inhibitor may advantageously be tested with several classes of enzyme activities, since it may display a strong specificity for a class or a subsclass of enzymes or on the contrary display some general inhibiting capacities. Candidate inhibitors to be tested by the method of the invention may be prepared by any method known by the skilled person in the art.

A further object of the present invention concerns a method for screening candidate beta-lactam agents (preferably antimicrobial agents) that are not hydrolyzed by said beta-lactamase activity, comprising the steps of:
(i) contacting a sample comprising said beta-lactamase activity (either within a biological cell or in a free form) with at least one candidate beta-lactam anti-microbial agent, in at least one electrochemical cell;
(ii) contacting the sample with a known substrate of said beta-lactamase activity;
(iii) detecting an impedance variation in said electrochemical cells of steps (i) and (ii) by collecting data points; and
(iv) comparing the impedance variations detected in step (iii);
   wherein said at least one candidate anti-microbial agent comprises a beta-lactam ring.

In one embodiment, the interaction of the at least one substrate with the beta-lactamase activity generates an impedance variation based on redox-activity. In another embodiment, said at least one substrate generates at least one redox-reaction when subjected to said enzyme activity.

In one embodiment, the impedance variation is detected with monitoring means as described hereinabove.

In another embodiment, the screening method of the invention aims at identifying new anti-microbial agents that could not be hydrolyzed by the tested specific beta-lactamase activity.

As a prerequisite, the candidate anti-microbial agents to be tested comprise compounds containing a beta-lactam ring. In an embodiment, the interaction of the candidate antimicrobial agent with the enzyme activity generates an impedance variation based in particular on redox-activity.

In the screening method of the invention, when an impedance variation is comparably detected in the presence of the known substrate of the tested beta-lactamase activity and in the presence of the candidate anti-microbial agent, it should then be inferred that the tested candidate anti-microbial agent is actually hydrolyzed by the beta-lactamase activity contained in the tested sample, and therefore that it could not be used as anti-microbial agent against the tested class of enzyme. On the contrary, if an impedance variation is detected in the presence of the known substrate of the tested beta-lactamase activity, but that no variation is detected in presence of the candidate anti-microbial agent, it should then be inferred that the tested candidate anti-microbial agent is resistant to hydrolysis by said beta-lactamase activity and is a promising anti-microbial agent against the tested class of enzyme.

In a particular embodiment, the identification and screening methods of the invention are advantageously performed with a sample containing a free enzyme having the tested enzyme activity, instead of being performed on a biological cell containing the said enzyme activity.

Another object of the invention is a method of diagnosing pathogen agents responsible for an infection or for detecting drug resistant pathogens.

In one embodiment, the method of the invention aimed at determining the origin of the resistance of a pathogen to a beta-lactam antimicrobial agent. In other words, the method of the invention aims at answering the following question: is a pathogen resistant to a beta-lactam antimicrobial agent because it expresses a beta-lactamase activity?

In one embodiment, the method of the invention is a method of determining if a beta-lactam antimicrobial agent may be useful to a patient. Indeed, if a patient is infected by a bacterial cell which expresses, as determined by the method of the invention, a beta-lactamase activity, then this patient will not benefit from the administration of a beta-lactam antimicrobial agent. Another antimicrobial agent may thus be used as a first choice in this patient.

In one embodiment, the method of the invention may be useful in the epidemiologic field. Indeed, a patient identified by the method of the invention as infected by a bacterial cell expressing a beta-lactamase activity may be quarantined, in order to avoid any contamination and therefore to avoid epidemic or pandemic.

According to one embodiment, the method of the invention comprises a step of comparing the impedance variation measured at step (ii) with the impedance variation measured in a reference method.

Preferably, said reference method does not comprise the step of contacting the sample with a substrate. Therefore, according to one embodiment, the method of the invention comprises a step of comparing the impedance variation measured in presence of the substrate with the impedance variation measured in absence of the substrate.

When an impedance variation is comparably detected in the presence and in absence of a substrate, it should then be inferred that the tested sample does not comprise a beta-lactamase activity. Similarly, if an impedance variation is detected in presence of the substrate but is no more detected in absence of this substrate, it should then be inferred that tested sample comprises a beta-lactamase activity.

In another embodiment, the method of the invention does not comprise a step of comparing the impedance variation measured at step (ii) with the impedance variation measured in a reference sample. Indeed, the Inventors have shown (see Examples) that very valuable results may also be obtained by analyzing only the results obtained in presence of the substrate (specificity of about 100%, sensitivity of about 95%). In one embodiment, this embodiment without negative control is particularly adapted when the method of the invention comprises a RESET step as described hereinabove.

Another object of the invention is a kit comprising an electrochemical cell containing an electrode as defined hereinabove, a microcontroller for analyzing the measured data, and optionally as least one buffer as described hereinabove.

Another object of the invention is a kit comprising a system as defined hereinabove, and optionally as least one buffer as described hereinabove.

Another object of the invention is an USB electrode reader to be used in the method of the present invention.

Another object of the invention is a wireless electrode reader to be used in the method of the present invention.

Another object of the invention is a data acquisition software (iOS, Windows, Linux or Android) for implementing the method of the present invention.

Another object of the invention is a data analysis software (iOS, Windows, Linux, or Android) for analyzing the data obtained by the method of the present invention.

The method of the present invention presents the following advantages over the methods of the prior art.
it is simple to implement;
it is safer as it requires only low volumes of bacterial suspensions, and/or only low number of bacterial cells;
it presents increased sensitivity and specificity as compared to the methods of the prior art (in particular, the method of the present invention may present a specificity of 100% and a sensitivity of 95% or more). Therefore, the method of the present invention allows detecting bacterial strains that were not detected with the methods of the prior art;
it results in a figure: therefore, the user does not need to interpret the result of the method of the invention, as may be the case with colorimetric methods of the prior art (the method of the invention is thus objective and not subjective).
Moreover, as the result of the method of the invention is a figure, it is traceable, and in particular it may be recorded on a data base, for example;
it is faster to implement: indeed, a result may be obtained in less than 1 hour, preferably in about 34 minutes, more preferably in at most 15 minutes, whereas the methods of the prior art may require until about 3 days for obtaining a result;
it may be implemented at room temperature (such as, for example, at a temperature ranging from about 15 to about 25° C.);
in certain embodiments, no lysis of the bacterial cell is needed;
it may be implemented directly on bacterial colonies (such as for example, bacterial colonies recovered from agar culture plates);
in certain embodiments, no negative control is required. In particular, no negative control is required when a RESET step is applied to the electrode of the invention before implementing the method of the invention;
the electrodes of the invention are reusable, which is of particular interest for environmental purposes (including limiting waste);
it allows detecting a beta-lactamase activity and to identify said beta-lactamase activity (i.e. a signature for a specific beta-lactamase activity may be identified according to the method of the present invention).

### BRIEF DESCRIPTION OF THE DRAWINGS

FIG. 1 is a drawing showing the redox equilibriums of polyaniline in vertical and the acid-base equilibriums thereof in horizontal. This polymer is known as stable and highly conductive in its emeraldine acid form.

FIG. 2 is a schematic drawing showing the architecture of a potentiostat and the corresponding feedback loop algorithm used for performing potentiometry measurements. ADC Target stands for Analog to Digital Converter Target. DAC stands for Digital to Analog Converter. IC1, IC2 and IC3 correspond to operational amplifiers that constitute the potentiostat. W corresponds to the working electrode. C corresponds to the counter electrode. R corresponds to the Reference electrode.

FIG. 3 is a chronogram showing the impedance measurement implemented in the examples of the present application. ADC stands for Analog to Digital Converter. DAC stands for Digital to Analog Converter.

FIG. 4 is a graph showing the global conductance measurement performed with the strain PEP119-KPC-2. The data are represented as exchanged charges in coulomb as a function of time.

FIG. 5 is a graph showing the global conductance measurement performed with the strain PEP175-NDM-1. The data are represented as exchanged charges in coulomb as a function of time.

FIG. 6 is a graph showing the global conductance measurement performed with the strain PEP77-CTX-M-15. The data are represented as exchanged charges in coulomb as a function of time.

FIG. 7 is a graph showing the global conductance measurement performed with the strain PEP141-OXA-48. The data are represented as exchanged charges in coulomb as a function of time.

FIG. 8 is a graph showing the summary of the impedance assays performed on 49 strains with the method of the invention in absence of cell lysis. The data are represented as arbitrary units as a function of the tested strain.

FIG. 9 is a graph showing impedance assays performed with the same electrode.

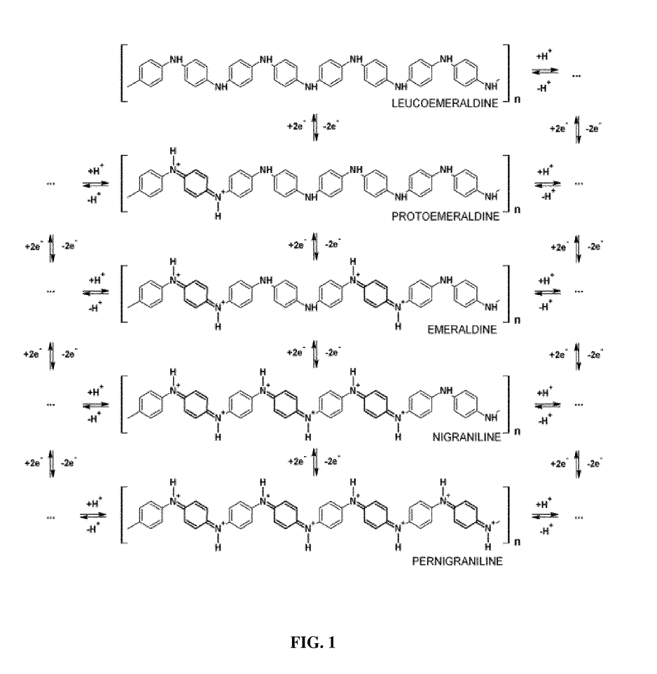

### EXAMPLES

The present invention is further illustrated by the following examples.

### Example 1: Potentiometric Assay

### Material and Methods

### Potentiostat

A potentiostat was prepared in accordance with the disclosure of WO 2011/082837.

### Electrodes

The prepared electrodes were composed of eight probes, disposed such that these probes could be inserted simultaneously in a line of wells of common 96 multi-well plateforms. These electrodes were obtained by classical printed circuit on board (PCB) realization techniques. The copper circuitry was protected by a solder mask varnish.

As polyaniline cannot be electro-synthesized on copper and as copper can be easily oxidized, all the electrodes area were coated with a screen printed carbon layer having a resistance of approximately 14-20 Ohm/square at 25 μm dry film thickness (Peeters S D 2841 HAL-IR).

Individual probes were composed of 3 electrodes round spots. The top spot had a diameter of one millimeter and constituted the working electrode on which polyaniline was electro-synthesized. The middle electrode was the reference electrode, had a diameter of one millimeter and was functionalized by applying a small spot of solid Ag/AgCl amalgam (Dupont 5874 Silver/Silver Chloride Composition—4 hours of curing at 80° C.) on top of the carbon layer. This solid Ag/AgCl reference electrode has been checked for its stability, repeatability and reliability in different measurement setups from pH=2 to 12. This reference displayed an electrode potential 100 mV higher (+300 mV vs. SHE) than a commercial Ag/AgCl reference electrode (+197 mV vs. SHE). The bottom electrode had a diameter of 1.5 millimeter and constituted the counter electrode. It had a bigger surface and was also covered with the Ag/AgCl amalgam in order to prevent it from being the current limitation against the working electrode.

Each of the eight probes of the prepared electrodes was assignable by multiplexers present on the potentiostat card. The reference and counter were common between probes regarding the potentiostat electronic circuit. The size of the instrument reached about 75 mm×55 mm×20 mm. The electrode probes were about 4 mm wide and could easily be inserted in common 96 multi-well platforms.

Polyaniline electro-polymerization was performed by using the potentiostat in coulometry on the eight electrode's probes placed in a row of a 96 multi-well platform. Each cell was filled with 300 μL of a 0.2M aniline/2M HCl aqueous solution. Electro-polymerization was performed up to 60 μC of charge on each working electrode (1 mm of diameter) at 890 mV against the solid Ag/AgCl reference electrode. After electro-synthesis, the electrode's probes were rinsed three times with distilled water, twice with 1M aqueous ammonia and finally three more times with distilled water. The electrodes were then dried using N₂ and stored in common 96 multi-well platforms before any test.

Aniline was distilled under reduced pressure prior to any experimentation.

All chemicals are purchased from Aldrich Chemicals. Electro-synthesis and measurements were all performed at room temperature (20° C.).

### Sample Preparation

Strains to be tested were grown overnight at 35° C. on a TSA (Trypticase Soy Agar 5% sheep blood) petri dish. Five calibrated inoculation loops (5×10 μL) of the tested strain were placed in a 2 mL eppendorf vial containing 500 μL of Tris-HCl 20 mmol/L lysis buffer (B-PERII, Bacterial Protein Extraction Reagent; Thermo Scientific Pierce, Rockford, Ill., USA) for 30 minutes to allow cell membrane lysis. Mixing was ensured by 1 minute of vortex agitation at start and every 10 minutes. After the thirty minutes, the sample was centrifuged for five minutes at 10000×g to allow biological solid debris sedimentation. An imipenem solution was prepared as fresh as possible during this centrifugation step because of its weak stability over time (4 hours at room temperature is considered as acceptable. Imipenem may nevertheless be prepared in advance and freezed a −20° C.) to a 3 mg/mL imipenem, 0.1 mM ZnSO₄ solution. Similarly, experiments were also performed with a solution of Tienam® at 6 mg/mL (comprising 3 mg/mL imipenem and 3 mg/mL of cilastatin)

### Results

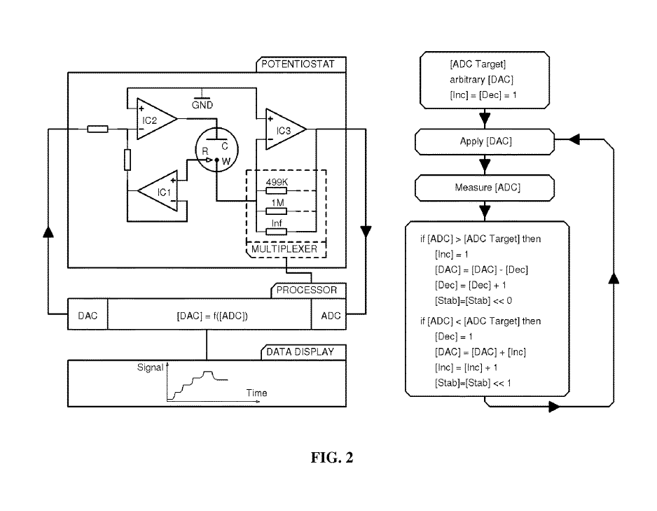

Electrochemical equilibrium potential was determined using the potentiostat and the method described in WO2011082837. As a reminder, and as depicted in FIG. 2 generating simple active measurements consisted in including a feedback loop between the input and the output of the potentiostat.

In the configuration of the experiment, the feedback loop was controlled, monitored and actuated with the aid of a micro-controller processor. The processor implemented a simple algorithm such as the one depicted in FIG. 2. This algorithm required some input data, the sought current as [ADC Target] (Analog to Digital Converter Target), an arbitrary initial guess for the potential to apply, the arbitrary [DAC] (Digital to Analog Converter), an incremental and a decremental variable, [Inc] and [Dec], having the DAC voltage resolution as initial value (1 mV in the present case). The processor applied the arbitrary working potential, arbitrary [DAC], on the potentiostat. This action resulted in a current flow in the electrochemical cell that is red after a desired amount of time as, [ADC]. The applied working potential, [DAC], was then increased or decreased with a variable step, to iteratively approach a desired current flow, [ADC Target]. If the targeted current was null ([ADC Target]=0), the applied potential simply tended to the equilibrium electrochemical potential of the working electrode.

For potentiometry, an infinite resistor was selected from the multiplexer. As a result, the current to voltage converter, IC3, limited the current between the counter and the working electrodes to the operational amplifier intrinsic current leak (typically 10-100 pA for common operational amplifiers). The current to voltage converter then reached a quasi-infinite gain and just mostly gave two extreme values indicating if the applied potential was higher or lower than the working electrode equilibrium potential. In this configuration, with [ADC Target]=0, the potentiostat acted as a discrete voltage comparator allowing to generate simple potentiometric detection with polyaniline. The use of an infinite resistor limited the current and avoided alteration of the polyaniline working electrode during the measurement.

The delay between the [DAC] application and the [ADC] measurement was fixed at 20 ms. When measurements were realized on the 8 probes, the potentiostat was multiplexed in order to select a different sensor for every iteration and the micro-controller managed 8 sets of the above mentioned parameters.

The electrode's sensors were placed in the 8 wells array containing a 200 μL solution 0.1 mM ZnSO₄ with or without 3 mg/mL imipenem and left to rest for 2 minutes while measuring the electrochemical potentials.

60 μL of pure BPERII or lysis extract were then mixed in the different wells of the array containing the electrodes.

A control without bacterial extract permitted to control the stability of the imipenem in the solution during the experiment and a control without imipenem confirmed that the signal was correlated with the hydrolysis of the carbapenem anti-microbial agents.

Measurement was followed during about 18 minutes. Electrodes were then calibrated with standard buffers at pH4, pH7, pH4 and pH7 for about 3 minutes each.

The different pH buffer solutions for calibration were obtained using FIXANAL recipes from the Riedel de Haen Company. For 1 liter aqueous solutions, the buffer at pH=4 contained 11.76 g of C₆H₈O₇.H₂O, 2.57 g of NaCl and 68 mL of NaOH (1 M), the buffer at pH=7 contained 3.52 g of KH₂PO₄ and 7.26 g of Na₂HPO₄.2H₂O, and the buffer at pH=10 contained 4.77 g of Na₂B₄O₇.10H₂O and 18.3 mL of NaOH (1 M).

In a typical experiment 2 strains were tested in parallel, the procedure could be described as below.

As the electrode counts eight probes, two strains (e.g. strain1 and strain2) were tested at the same time. From an array of 8 wells numbered from 1 to 8, wells 1, 2, 4, 5, 7, 8 contained 200 μL of the 3 mg/mL imipenem, 0.1 mM ZnSO₄ solution. Wells 3 and 6 contained 200 μL of the 0.1 mM ZnSO₄ solution without imipenem.

Another 8 wells array was prepared with 70 μL pure BPERII in wells 1 and 2, 70 μL of strain1 lysis extract in wells 3, 4 and 5 and 70 μL of strain2 lysis extract in wells 6, 7 and 8. 60 μL were taken from this array using an 8 channels micropipette. These 60 μL were mixed in the array containing the electrode and the measurements were carried out.

As the carbapenem hydrolysis is known to produce a pH variation, potentiometric experiments were performed on 2 carbapenemases-producing strains (PEP119 and PEP175) that had already been tested as clearly positive by the CarbaNP-test (Nordmann-Poirel test). PEP119 and PEP175 are Klebsiella pneumonia strains that are respectively expressing KPC-2 and NDM-1 genes.

An increase of polyaniline potential upon the addition of the lysis extract to imipenem was observed when compared to the extract measured without imipenem (value after 6 minutes for KPC-2=+50 mV and for NDM-1=+20 mV). However, as a +60 mV variation corresponds to a dropdown of one unit pH variation, the signals seemed pretty low for such strains when compared with the extent of their signals obtained with the CarbaNP-test. This observation suggested that the electrodes were undergoing two opposite effects in potential, and that some reduction process could lower the potential while acidity would increase it. This reduction phenomenon was clearly evidenced by immediately testing the electrodes with known pH calibration standards right after the potentiometric test.

The electrodes were clearly shown to be reduced in presence of the tested strains extracts and of the beta-lactam, with a variation in potential of about −120 mV at pH=4 and about −50 mV at pH=7 for both strains. This means that the signal observed before pH calibration, without any pH calibration standard, was reflecting a pH variation combined with reduction of the electrodes (data not shown).

The specific observation of the pH variation could be obtained by calibrating the signals with the signals for the pH standards. However, the effect of the calibration on the early stages of the signal acquisition, immediately after the addition of the strain extract, is possibly erroneous as the kinetics of the redox and acid-base reaction were not known. No reduction of the electrode and no decrease of pH could be observed for the strains expressing no carbapenemase. Similarly, no reduction of the electrode and no decrease of pH could be observed for the strains expressing the weak carbapenem hydrolyser OXA-48.

The potentiometric tests clearly indicated that the electrode reduction and acidification have opposite effects on the potential: acidity increases the potential whereas reduction decreases it.

### Example 2: Impedance Assay

### Materials and Methods

### Electrodes

Electrodes for use in the impedance assays were prepared as disclosed previously in Example 1.

Due to instrumentation limitation, such as natural noise, unlike potentiometry, impedance measurement of the electrochemical cell necessitated to flow the lowest measurable current through the polyaniline working electrode. In order to minimize the electrode alteration, this sampling had to be as short as possible in time and as close as possible to the equilibrium electrochemical potential of the electrode.

For polyaniline impedance measurements, the electrochemical potential was first determined with the above mentioned potentiometry method. However, to ensure that the measured potential was at the equilibrium and stable in time, an algorithm was implemented in order to check for stability in time. Using the previously described potentiometric method, once the equilibrium potential was reached and due to the finite DAC resolution, the algorithm would have in any case kept on searching the targeted current. In an ideal case, this would have resulted in 1 mVₚₚ square oscillations. The resulting pulse train could then be red during time to be interpreted as a binary number (1 for up, 0 for down) that is bit-shifted every iteration of the algorithm, as described by the [Stab] parameter in FIG. 2. In this work, [Stab] is defined as a 32 bit integer that is initially null.

As various stability patterns can arise as a function of the electrode's impedance, five patterns have been considered as indicative of the equilibrium electrochemical potential stability. The corresponding binary pulse train numbers and their corresponding integer conversions are indicated in Table 1.

TABLE 1
Train pulse's binary numbers for stability and their conversions
to integers.

| Binary pulse train numbers | Corresponding integer |
|---|---|
| 10101010101010101010101010101010 | 2863311530 |
| 11001100110011001100110011001100 | 3435973836 |
| 11100011100011100011100011100011 | 3817748707 |
| 11110000111100001111000011110000 | 4042322160 |
| 11111000001111100000111110000011 | 4164816771 |

Once the [Stab] parameter equals one of these numbers, the electrode is considered as stable and an impedance measurement can be initiated in a relatively optimal condition. After this measurement, the [Stab] parameter is reset to a null value.

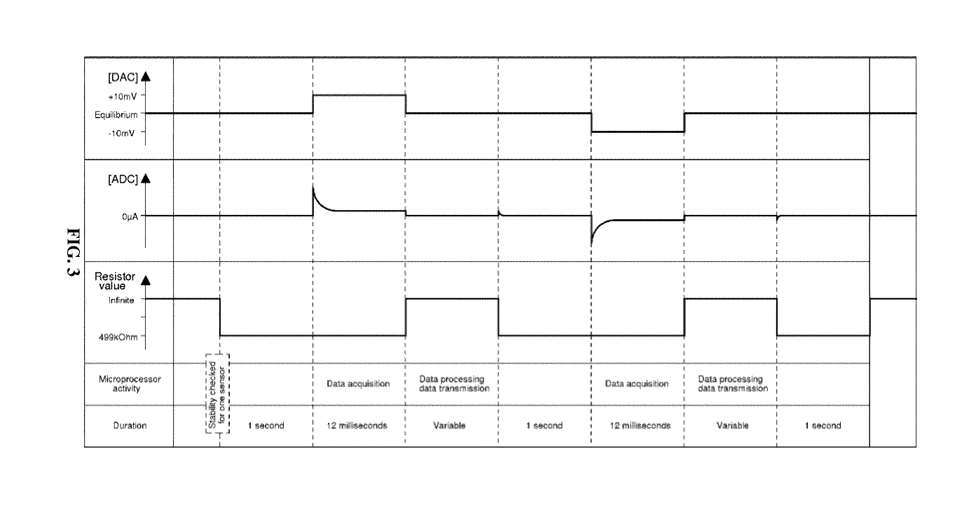

The chronogram in FIG. 3 depicts the different sequences of an impedance experiment: once stability is met for one of the sensor, the 499 kOhm resistor is selected by the multiplexer and the last determined equilibrium potential is applied via the [DAC] parameter for 1 second to settle the corresponding probe. After that time, the potential is raised by 10 mV at the DAC maximum speed and the current transient response is measured at a 34487 Hz sampling rate during 11.6 ms. After that time, the infinite resistor is selected by the multiplexer and the 400 measured data points are transferred to the computer. After this first data transfer, the 499 kOhm resistor is selected by the multiplexer and the equilibrium potential is applied via the [DAC] parameter for 1 second to settle the electrode again. After that time, the potential is now decreased by 10 mV at the DAC maximum speed and the current transient response is measured at a 34487 Hz sampling rate during 11.6 ms again. After that time, the infinite resistor is selected by the multiplexer and the new 400 data points are transferred to the computer.

To be complete, regarding the simplified scheme of FIG. 2 a 10 pF capacitor is added in parallel to the 499 kOhm resistor to reduce noise.

As depicted in the chronogram of FIG. 3, the resulting current transients are generally decays in electrochemistry. These decays hide many of the electrode interface properties such as solution and interface conductance, solution double layer capacitance, etc.

The amount of data was drastically reduced through pretreatment. In the present case, the two decays were usually completely identical owing the stability and the low amplitude of the excitation. These were then, after a change in sign for the second transient, averaged to a single positive decay. It was chosen to calculate 5 contiguous integrals of 80 data each in order to allow different types of modeling and to recover up to five parameters.

In the presented results, the simplest operation which is the sum of these five integrals, corresponding after simple Ohm's law calculation to a global conductance, largely sufficed to obtain very high specificities and sensitivities regarding the present test for detecting carbapenemase producing Enterobacteriaceae.

In the presented work, the data is represented as exchanged charges in coulomb as a function of time.

Concerning the stability parameters, as stability occurs randomly, data were generated at random times. As a consequence, data comparison between the different probes of one electrode necessitated interpolating the data to a constant time base. In this work, cubic-spline interpolations, data treatments and plots were obtained using GNU Octave 3.6.4.

### Sample Preparation

The present impedance test was performed in parallel on the same bacterial extract to the CarbaNP-test according to the procedure published by Nordmann et al. and the results were compared. In brief, one calibrated dose (10 μL) of the tested strain directly recovered from the antibiogram was suspended in a Tris-HCl 20 mM lysis buffer (B-PERII, Bacterial Protein Extraction Reagent; Thermo Scientific Pierce, Rockford, Ill., USA), vortexed for 1 minute and further incubated at room temperature for 30 minutes. This bacterial suspension was centrifuged at 10000×g at room temperature for 5 minutes. Thirty μL of the supernatant, corresponding to the enzymatic bacterial suspension, was mixed in a 96-well tray with 100 μL of a 1 mL solution made of 3 mg of imipenem monohydrate (Sigma, Saint-Quentin Fallavier, France), pH 7.8, phenol red solution, and 0.1 mM ZnSO₄ (Merck Millipore, Guyancourt, France).

Sixty μL of the supernatant was mixed with 200 μL of a 3 mg/L of imipenem solution containing 0.1 mM of ZnSO₄. The electrodes were immersed in the solution and the signal was collected during a maximum of one hour duration.

Alternatively, in order to improve the turnaround time of the test, another procedure was evaluated. In this protocol, 10 μL calibrated dose of bacteria was suspended in 100 μL of saline solution (75 mM MgCl₂75 mM CaCl₂), 30 μL of the suspension is transferred to 60 μL of a solution containing 0.1 mM ZnSO₄ with 3 mg/mL of imipenem) and 50 μL of the suspension was put directly on the electrode without previous incubation and centrifugation. The signal was collected during a maximum of 30 minutes.

A control without bacterial extract permitted to control the stability of the imipenem in the solution during the experiment and a control without imipenem confirmed that the signal is correlated with the hydrolysis of the carbapenem anti-microbial agents.

### Strains

A total of 121 Enterobacteriaceae, were tested (see table 2 below). This collection included 53 isolates carrying different β-lactamase genes (including carbapenemases) expressed in various species that had been characterized, 10 from the External quality control of NEQAS for carbapenemases detection 2013 (Labeled Naps) and 58 isolates (Labeled NRC) OXA-48 producers (n=39) and non-carbapenemase producers (n=19), for which the presence of beta-lactamases and carbapenemases was assessed according to ISO15189 certified end-point PCR.

TABLE 2
Description of the 121 tested Enterobacteriaceae strains.

| | Ref. | | | | | | | | Minor | Other |
|---|---|---|---|---|---|---|---|---|---|---|
| Origin | number | Species | Carbapenemase | TEM | SHV | OXA | CTX-M | AmpC | ESBL | resistances |
| NRC | 20130343 | E. coli | OXA-48 like | TEM | — | OXA-1 | — | | | |
| NRC | 20130348 | K. pneumoniae | OXA-48 | TEM | SHV | OXA-1 | CTX- | | | |
| | | | | | | | M G1 | | | |
| NRC | 20130355 | K. pneumoniae | OXA-48 like | TEM | SHV | OXA-1 | CTX- | | | |
| | | | | | | | M G1 | | | |
| NRC | 20130364 | C. freundii | OXA-48 like | TEM | — | — | — | | | |
| NRC | 20130413 | K. pneumoniae | OXA-48 like | — | SHV | — | — | | | |
| NRC | 20130429 | K. pneumoniae | OXA-48 like | TEM | SHV | OXA-1 | CTX- | | | |
| | | | | | | | M G1 | | | |
| NRC | 20130446 | K. pneumoniae | — | TEM | SHV | — | CTX- | | | |
| | | | | | | | M G2 | | | |
| NRC | 20130450 | K. pneumoniae | OXA-48 | TEM | SHV | OXA-1 | CTX- | | | |
| | | | | | | | M G1 | | | |
| NRC | 20130451 | K. pneumoniae | OXA-204 | TEM | SHV | OXA-1 | CTX- | | | |
| | | | | | | | M G1 | | | |
| NRC | 20130455 | E. coli | — | TEM | — | — | — | CMY-2 | | |
| NRC | 20130471 | E. coli | — | TEM | SHV | — | CTX- | | | |
| | | | | | | | M-15 | | | |
| NRC | 20130484 | K. pneumoniae | — | TEM | SHV | OXA-1 | CTX- | | | |
| | | | | | | | M G1 | | | |
| NRC | 20130485 | E. aerogenes | — | TEM | — | — | — | | | |
| NRC | 20130493 | K. pneumoniae | — | — | SHV | OXA-1 | CTX- | DHA | | |
| | | | | | | | M G1 | | | |
| NRC | 20130515 | E. coli | — | TEM | — | OXA-1 | CTX- | | | |
| | | | | | | | M G1 | | | |
| NRC | 20130523 | E. cloacae | — | — | SHV | — | CTX- | | | |
| | | | | | | | M G9 | | | |
| NRC | 20130524 | K. pneumoniae | — | TEM | SHV | OXA-1 | CTX- | | | |
| | | | | | | | M G1 | | | |
| NRC | 20130530 | K. pneumoniae | OXA-48 like | — | SHV | OXA-1 | CTX- | — | | |
| | | | | | | | M G1 | | | |
| NRC | 20130531 | K. pneumoniae | OXA-48 like | TEM | SHV | OXA-1 | CTX- | | | |
| | | | | | | | M G1 | | | |
| NRC | 20130536 | K. pneumoniae | OXA-48 like | — | SHV | — | — | | | |
| NRC | 20130539 | K. pneumoniae | OXA-48 like | TEM | SHV | OXA-1 | CTX- | | | |
| | | | | | | | M G1 | | | |
| NRC | 20130540 | K. pneumoniae | OXA-48 like | TEM | SHV | OXA-1 | CTX- | | | |
| | | | | | | | M G1 | | | |
| NRC | 20130545 | K. pneumoniae | OXA-48 like | TEM | SHV | OXA-1 | CTX- | | | |
| | | | | | | | M G1 | | | |
| NRC | 20130546 | K. pneumoniae | OXA-48 like | TEM | SHV | — | — | | | |
| NRC | 20130549 | E. coli | OXA-48 like | TEM | — | — | — | | | |
| NRC | 20130550 | K. pneumoniae | OXA-48 like | TEM | SHV | — | — | | | |
| NRC | 20130551 | E. coli | OXA-48 like | TEM | — | — | — | | | |
| NRC | 20130553 | S. marcescens | OXA-48 like | — | — | — | — | | | |
| NRC | 20130557 | K. pneumoniae | OXA-48 like | TEM | SHV | OXA-1 | CTX- | | | |
| | | | | | | | M G1 | | | |
| NRC | 20130561 | K. pneumoniae | OXA-48 like | TEM | SHV | OXA-1 | CTX- | | | |
| | | | | | | | M G1 | | | |
| NRC | 20130562 | E. coli | — | TEM | SHV | OXA-1 | CTX- | — | | |
| | | | | | | | M G1 | | | |
| NRC | 20130563 | K. pneumoniae | OXA-48 like | — | SHV | OXA-1 | — | | | |
| NRC | 20130572 | E. coli | OXA-48 like | — | — | — | — | | | |
| NRC | 20130573 | K. pneumoniae | OXA-48 like | TEM | SHV | OXA-1 | CTX- | | | |
| | | | | | | | M G1 | | | |
| NRC | 20130625 | K. pneumoniae | — | TEM | SHV | OXA-1 | CTX- | | | |
| | | | | | | | M G1 | | | |
| NRC | 20130629 | K. pneumoniae | OXA-48 like | TEM | SHV | OXA-1 | CTX- | | | |
| | | | | | | | M G1 | | | |
| NRC | 20130675 | E. cloacae | — | — | — | — | — | | | |
| NRC | 20130700 | E. cloacae | OXA-48 like | — | — | — | CTX- | | | |
| | | | | | | | M G9 | | | |
| NRC | 20130707 | E. coli | OXA-48 like | — | — | OXA-1 | CTX- | | | |
| | | | | | | | M G1 | | | |
| NRC | 20130708 | K. pneumoniae | OXA-48 like | TEM | SHV | OXA-1 | CTX- | | | |
| | | | | | | | M G1 | | | |
| NRC | 20130716 | E. coli | OXA-48 like | — | SHV | — | CTX- | | | |
| | | | | | | | M G1 | | | |
| NRC | 20130744 | K. pneumoniae | OXA-48 like | TEM | SHV | OXA-1 | CTX- | | | |
| | | | | | | | M G1 | | | |
| NRC | 20130746 | K. pneumoniae | OXA-48 like | TEM | SHV | OXA-1 | CTX- | | | |
| | | | | | | | M G1 | | | |
| NRC | 20130747 | S. marcescens | OXA-48 like | | | | | | | |
| NRC | 20130748 | K. pneumoniae | OXA-48 like | TEM | SHV | — | — | | | |
| NRC | 20130749 | E. coli | OXA-48 like | TEM | — | — | — | | | |
| NRC | 20130750 | K. oxytoca | — | | | | | | | |
| NRC | 20130751 | S. marcescens | — | — | — | — | — | | | |
| NRC | 20130752 | E. coli | OXA-48 like | TEM | — | — | — | | | |
| NRC | 20130753 | K. pneumoniae | OXA-48 like | — | SHV | OXA-1 | CTX- | | | |
| | | | | | | | M G1 | | | |
| NRC | 20130760 | K. pneumoniae | — | — | SHV | OXA-1 | CTX- | | | |
| | | | | | | | M G1 | | | |
| NRC | 20130762 | K. pneumoniae | OXA-48 like | TEM | SHV | OXA-1 | CTX- | | | |
| | | | | | | | M G1 | | | |
| NRC | 20130790 | K. pneumoniae | — | — | SHV | OXA-1 | CTX- | | | |
| | | | | | | | M G1 | | | |
| NRC | 20130796 | K. pneumoniae | OXA-48 like | TEM | SHV | OXA-1 | CTX- | | — | |
| | | | | | | | M G1 | | | |
| NRC | 20130828 | K. pneumoniae | — | TEM | SHV | OXA-1 | CTX- | | | |
| | | | | | | | M G1 | | | |
| NRC | 20130877 | K. pneumoniae | OXA-48 like | TEM | SHV | — | CTX- | | | |
| | | | | | | | M G1 | | | |
| NRC | 20130906 | K. pneumoniae | — | TEM | SHV | OXA-1 | CTX- | | | |
| | | | | | | | M G1 | | | |
| NRC | 20130454 | M. morganii | — | — | — | — | | | | |
| NEQAS | NEQAS | K. pneumoniae | KPC | | | | | | | |
| | 1940 | | | | | | | | | |
| NEQAS | NEQAS | E. cloacae | NDM | | | | | | | |
| | 1941 | | | | | | | | | |
| NEQAS | NEQAS | K. pneumoniae | KPC | | | | | | | |
| | 1942 | | | | | | | | | |
| NEQAS | NEQAS | K. pneumoniae | OXA-48 | | | | | | | |
| | 1943 | | | | | | | | | |
| NEQAS | NEQAS | K. pneumoniae | KPC | | | | | | | |
| | 1944 | | | | | | | | | |
| NEQAS | NEQAS | K. pneumoniae | VIM | | | | | | | |
| | 1945 | | | | | | | | | |
| NEQAS | NEQAS | K. pneumoniae | NDM | | | | | | | |
| | 1946 | | | | | | | | | |
| NEQAS | NEQAS | K. pneumoniae | IMP | | | | | | | |
| | 1947 | | | | | | | | | |
| NEQAS | NEQAS | K. pneumoniae | NDM | | | | | | | |
| | 1948 | | | | | | | | | |
| NEQAS | NEQAS | E. aerogenes | — | | | | | | | |
| | 1949 | | | | | | | | | |
| Tempo | PEP006 | C. freundii | — | TEM-1 | — | — | — | CMY- | — | |
| | | | | | | | | 2 like | | |
| Tempo | PEP007 | E. coli | — | TEM-1 | SHV- | — | — | DHA-7 | — | |
| | | | | | 12 | | | | | |
| Tempo | PEP008 | E. asburiae | — | TEM-1 | SHV- | — | — | ACT | — | |
| | | | | | 12 | | | | | |
| Tempo | PEP009 | P. mirabilis | — | — | — | — | CTX- | — | — | |
| | | | | | | | M-2 | | | |
| Tempo | PEP010 | E. aerogenes | — | TEM- | SHV- | — | — | | — | |
| | | | | 24 | 2a | | | | | |
| Tempo | PEP012 | P. stuartii | — | — | — | — | — | — | — | |
| Tempo | PEP016 | E. coli | — | TEM-1 | SHV- | — | — | ACC-1 | — | |
| | | | | | 2a | | | | | |
| Tempo | PEP018 | C. amalonaticus | | — | — | — | — | | GES-7 | |
| Tempo | PEP025 | S. marcescens | — | TEM-1 | — | — | CTX- | | — | |
| | | | | | | | M-3 | | | |
| | | | | | | | ou 22 | | | |
| Tempo | PEP027 | Salmonella | — | TEM-1 | — | OXA- | CTX- | | — | |
| | | | | | | 10 | M-15 | | | |
| Tempo | PEP029 | C. braakii | VIM-1 | — | — | — | — | | — | |
| Tempo | PEP031 | E. coli | NDM-1 | TEM-1 | — | OXA-1 | CTX- | CMY- | — | |
| | | | | | | | M-15 | 58 | | |
| Tempo | PEP032 | M. morganii | NDM-1 | TEM- | — | OXA-1 | CTX- | DHA | — | |
| | | | | | | | M-15 | | | |
| Tempo | PEP033 | E. cloacae | NDM-1 | TEM-1 | SHV- | OXA-1 | CTX- | MIR | — | |
| | | | | | 12 | | M-15 | | | |
| Tempo | PEP041 | K. pneumoniae | — | TEM- | SHV- | OXA-2 | — | ACT-1 | — | |
| | | | | 10 | 11 | | | | | |
| Tempo | PEP061 | K. pneumoniae | — | — | SHV- | — | CTX- | | — | |
| | | | | | 76 | | M-14 | | | |
| Tempo | PEP070 | K. pneumoniae | VIM-1 | TEM-1 | | OXA- | | | — | |
| | | | | | | 10 | | | | |
| Tempo | PEP075 | K. pneumoniae | KPC-2 | TEM-1 | SHV- | OXA-9 | | | — | |
| | | | | | 11 | tronqueet | | | | |
| | | | | | and - | OXA | | | | |
| | | | | | 12 | G1 | | | | |
| Tempo | PEP077 | E. coli | — | TEM-1 | — | — | CTX- | | — | ArmA |
| | | | | | | | M-15 | | | |
| Tempo | PEP084 | C. braakii | GES-6 | — | — | — | — | | GES-7 | |
| Tempo | PEP101 | S. marcescens | VIM-4 | TEM | — | — | — | | — | |
| | | | | WT | | | | | | |
| Tempo | PEP102 | A. caviae | VIM-4 | — | — | OXA-1 | — | | — | |
| Tempo | PEP108 | K. pneumoniae | — | TEM-1 | SHV- | OXA-1 | CTX- | | — | |
| | | | | | 28 | | M-15 | | | |
| Tempo | PEP119 | K. pneumoniae | KPC-2 | TEM-1 | SHV- | | | | — | |
| | | | | | 11 | | | | | |
| | | | | | and - | | | | | |
| | | | | | 12 | | | | | |
| Tempo | PEP124 | K. oxytoca | VIM-1 | — | — | — | | | — | |
| Tempo | PEP126 | K. pneumoniae | VIM-27 | — | SHV- | — | | | — | |
| | | | | | 11 | | | | | |
| Tempo | PEP130 | K. pneumoniae | KPC-2 | TEM-1 | SHV- | | | | — | |
| | | | | | 11 et - | | | | | |
| | | | | | 12 | | | | | |
| Tempo | PEP131 | P. vermicola | VIM-1 | — | — | — | | | — | |
| Tempo | PEP134 | K. pneumoniae | NDM-1 | TEM-1 | SHV- | OXA-1 | CTX- | | — | |
| | | | | | 12 | and | M-15 | | | |
| | | | | | | OXA-9 | | | | |
| Tempo | PEP135 | E. coli | NDM-1 | TEM-1 | — | OXA- | — | CMY- | — | |
| | | | | | | 10 | | 16 | | |
| Tempo | PEP136 | K. pneumoniae | OXA-48 | TEM-1 | SHV- | — | — | | — | |
| | | | | | 11 | | | | | |
| Tempo | PEP137 | K. pneumoniae | OXA-48 | TEM-1 | SHV- | OXA-1 | CTX- | | — | |
| | | | | | 11 | | M-15 | | | |
| Tempo | PEP138 | K. pneumoniae | OXA-48 | TEM-1 | SHV- | OXA-1 | CTX- | | — | |
| | | | | | 11 | | M-15 | | | |
| Tempo | PEP140 | K. pneumoniae | OXA-48 | — | SHV- | — | — | | — | |
| | | | | | 11 | | | | | |
| Tempo | PEP141 | E. coli | OXA-48 | TEM-1 | — | — | CTX- | | — | |
| | | | | | | | M-27 | | | |
| Tempo | PEP143 | E. cloacae | OXA-48 | — | SHV- | — | CTX- | | — | |
| | | | | | 12 | | M-9 | | | |
| Tempo | PEP144 | E. cloacae | VIM-31 | — | — | — | CTX- | MIR | — | |
| | | | | | | | M-9 | | | |
| Tempo | PEP156 | E. coli | OXA-48 | | | | | | — | |
| Tempo | PEP157 | C. freundii | OXA-48 | | | | | | — | |
| Tempo | PEP158 | K. oxytoca | OXA-48 | | | | | | — | |
| Tempo | PEP163 | K. pneumoniae | KPC-2 | | | | | | — | |
| Tempo | PEP164 | K. pneumoniae | KPC-2 | | | | | | — | |
| Tempo | PEP165 | K. pneumoniae | KPC-2 | | | | | | — | |
| Tempo | PEP166 | K. pneumoniae | KPC-2 | | | OXA-9 | | | — | |
| Tempo | PEP167 | K. pneumoniae | KPC-2 | | | | | | — | |
| Tempo | PEP175 | K. pneumoniae | NDM-1 | | | | | | — | |
| Tempo | PEP177 | K. pneumoniae | NDM-1 | | | | | | — | |
| Tempo | PEP196 | K. pneumoniae | — | — | SHV- | — | — | DHA-1 | — | |
| | | | | | 11 | | | | | |
| Tempo | PEP198 | K. pneumoniae | OXA-48 | TEM-1 | — | — | CTX- | DHA | — | |
| | | | | | | | M-15 | | | |
| Tempo | PEP199 | E. cloacae | OXA-48 | TEM-1 | SHV- | — | — | DHA | — | |
| | | | | | 12 | | | | | |
| Tempo | PEP223 | E. coli | — | TEM- | — | — | — | ESAC | | |
| | | | | 30 | | | | | | |
| Tempo | PEP224 | E. coli | NDM-5 | TEM | — | OXA-1 | — | CMY- | | |
| | | | | | | | | 2 like | | |
| Tempo | PEP225 | E. cloacae | GIM-1 | | | | | | | |

For impedance measurements, the two phenomena, reduction and acidification, do act together complementarily. Indeed, besides its electrochemical potential dependence, polyaniline conductivity is also varying upon pH and redox activities, but by many orders of magnitude. Polyaniline is generally obtained in a slightly oxidized form after its electro-synthesis, its conductivity is not at its highest regarding its redox state. Reduction will then tend polyaniline to return to its emeraldine form which is the most conductive. In addition, the previous potentiometric experiments have clearly shown that acidity can vary up to two orders of magnitude (−2 pH units) and the acid form of the emeraldine is known to be even more conductive. As an overall consequence, conductivity increases as a function of both acidification and reduction. Impedance of the polyaniline electrodes was measured instead of the potential.

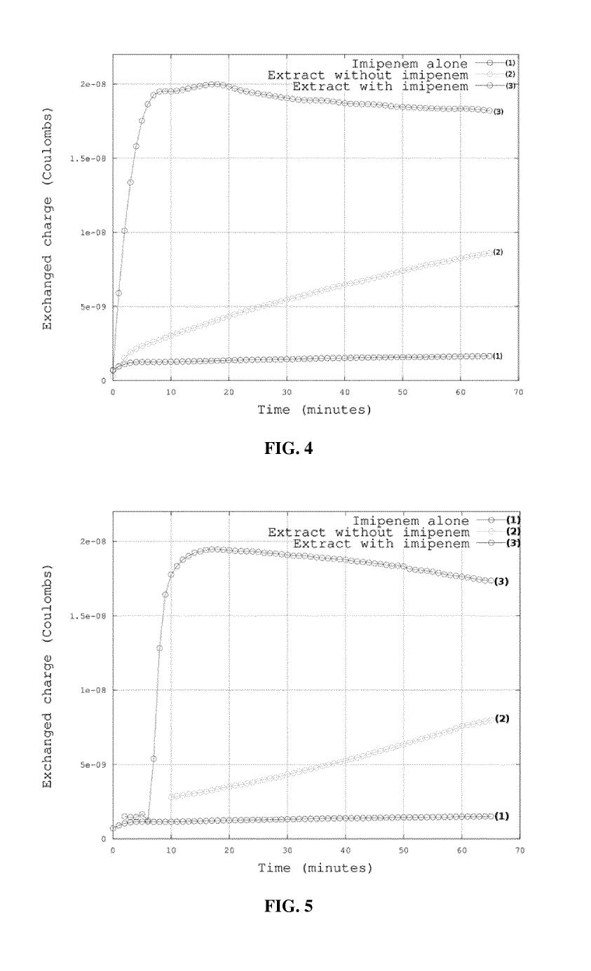

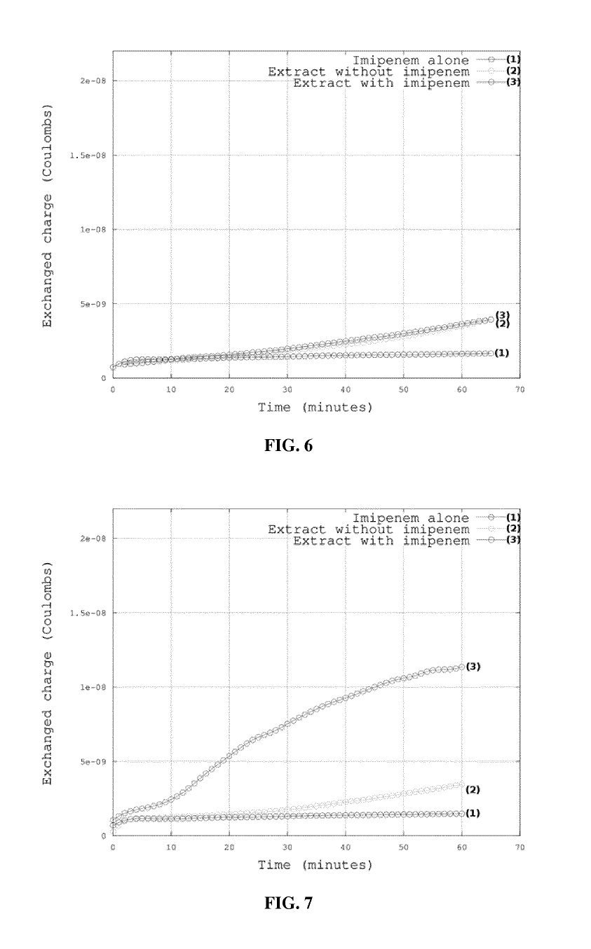

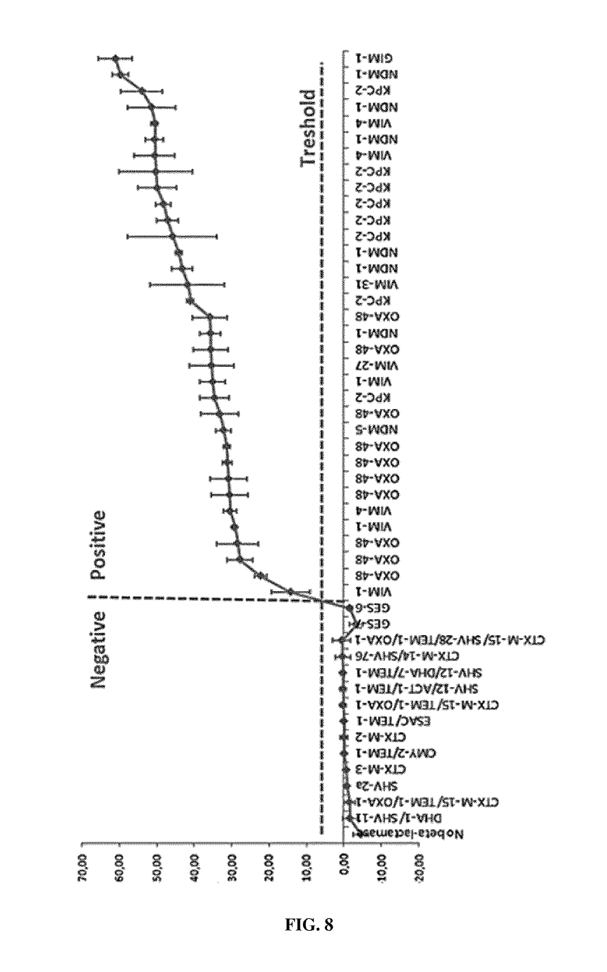

FIG. 4 and FIG. 5 represent the results obtained on the same strains, respectively PEP119 and PEP175, for impedance measurements. Signals in the presence of the imipenem are very high when compared to the extract alone. More interestingly, FIG. 6 and FIG. 7 represent the signals obtained for the PEP77 a CTX-M-15 (non carbapenemase)-producing strain and the PEP141 that produces the carbapenemase OXA-48. While no difference between the curves with and without imipenem in impedance is observed for PEP77, the imipenem curve is clearly different for PEP141 the OXA-48 producer.

The impedance method dramatically improved the detection of carbapenemases producers and thus constitutes a new diagnostic tool for the detection of the carbapenemases.

In a first embodiment, the results were obtained after 30 minutes of lysis using B-PERII, followed by a centrifugation step. The electrode was then plunged into 200 μL of the reaction mix including the protein extract.

In a further embodiment, the lysis incubation and the centrifugation steps were eliminated. Further, instead of plunging the electrode into the reaction mixture, 50 μL of the reaction mixture were deposited directly on the electrode to reduce awkward manipulations.

In a bulk solution process, as in the CarbaNP-test, the bacteria cells have to be lyzed in order to release the beta-lactamases and allow their accessibility for reaction with the imipenem substrate and the subsequent colorimetric reaction. In electrochemistry, these reactions are specifically observed at the interface between the electrode and the reaction mixture, thus it is mainly the reactions taking place at the interface that interplays with the electrode. Polyaniline was found to be a sufficiently good mediator to eliminate the need for the detergent lysis extraction. Further, additional experiments demonstrated that the presence of the carbapenemase activity (including OXA-48 activity) could also be detected, even in absence of salts.

Nevertheless, an appropriate salt concentration facilitates the beta-lactamase accessibility at the contact of the electrode. More than 10 different salt mixtures were tested at various concentrations and in different combinations (CaCl₂, MgCl₂, MnCl₂, MgSO₄, NH₄Cl, NaCl, KCl, CaSO₄, ZnCl₂ and the combination of CaCl₂ and MgCl₂): the carbapenemase-producing strains were detected with all the tested salt mixtures, but the best results were obtained with the mixture described hereinabove (i.e., with the combination of CaCl₂ and MgCl₂).

### Example 3: Comparison Between CarbaNP-Test and the Impedance Test (with and without Cell Lysis)

Results obtained on collection strains with the CarbaNP-test, the impedance test, comprising the lysing step, and the impedance test in absence of lysis step were compared.

In a first set of experiment, all the 53 collection strains (“Tempo” labeled strains in table 2) were analyzed using the same B-PER extraction procedure as described above. The supernatant was used in parallel for carbapenemases detection with the CarbaNP-test and the impedance test of the invention with cell lysis. The same strains are also evaluated with the impedance test without cell lysis using the new procedure without incubation and centrifugation The B-PER lysis buffer was replaced by a non-buffered salt solution as described above and the bacterial suspension was immediately analyzed with the impedance test. Contrarily to the first set of experiment, the electrode was not plunged into the solution but about 50 μL of the reaction mix were directly deposited on the electrode. Advantageously, the detection test of the invention can be performed at Room temperature, whereas other known detection tests (e.g. the CarbaNP-test) require the mixture to be incubated at 35-37° C., thereby preventing a continuous monitoring of the results.

This simplified step eliminated the 30 minutes incubation and the preliminary centrifugation step. Skipping the centrifugation step was already proposed by Nordmann et al. in a simplified process but using the bacterial suspension renders the interpretation of the results more difficult because of the turbidity of the medium and the observation of a higher rate of undetermined results (negative control without imipenem becoming yellow or orange).

With the impedance test of the invention, with and without cell lysis, a resulting curve was automatically calculated by subtracting the signal obtained without imipenem from the signal with imipenem. When the resulting curve crossed a determined threshold that is function of time, the strain was considered as a carbapenemase producer. The strains without carbapenemase did not cross this threshold.

As could be seen in table 3 below, the CarbaNP-test presents a sensitivity, specificity positive predictive value (PPV) and negative predictive value (NPV) of 89.2, 100, 100 and 80% respectively. These values are of 97.3, 100, 100 and 94.12% with the conductivity test of the present invention with cell lysis. No false positive results were observed with the 3 techniques and the weak and rare carbapenemase GES-6 was not detected by any of the tested methods. More interesting is the fact that one OXA-48-producer was not detected by the CarbaNP-test but was indeed detected by the impedance tests of the invention, with and without cell lysis. The CarbaNP-test is reported for its weakness for detecting this important resistant trait. The time to results after incubation and centrifugation was about 45 min for the CarbaNP-test and the impedance test with cell lysis and 30 min for the impedance test without cell lysis.

TABLE 3
Comparison of the 3 tests on 53 collection strains.

| | | Impedance test with | Impedance test |
|---|---|---|---|
| | Carba NP-test | cell lysis | without cell lysis |
| Sensitivity (%) | 89.19 | 97.3 | 97.3 |
| Specificity (%) | 100 | 100 | 100 |
| PPV (%) | 100 | 100 | 100 |
| NPV (%) | 80 | 94.12 | 94.12 |

The values obtained for the 3 tests were comparable but traceability of the results was significantly improved with the impedance tests of the invention. These tests also dramatically reduced the time to result and the hands-on time.

All NDM, KPC, VIM or IMP-producing strains were detected within 5 minutes of incubation either by the CarbaNP-test or the impedance tests of the invention. The evaluation of the time to positivity was nevertheless not as easy to evaluate precisely for the CarbaNP-test as it is for the impedance tests of the invention. In the CarbaNP-test, the result is indeed evaluated by the operator naked eye whereas the result of the impedance tests is a quantified, computed and traceable signal. Further, the maximum time of incubation with imipenem (which mean the time require to confirm that a strain is really not a carbapenemase-producer) is 2 hours for CarbaNP-test, vs. 1 hour for the impedance test with cell lysis and 30 min for the impedance test without cell lysis.

To confirm these results, the 3 tests were also compared on 58 isolates (labeled NRC) OXA-48 producers (n=39) and non-carbapenemase-producers (n=19) received from Belgian laboratories. On Table 4 below, it is shown that the CarbaNP-test presents a sensitivity, specificity, PPV and NPV of 78.38, 100, 100 and 68% respectively. These values were almost identical with 76.92, 100, 100 and 67.86% for the impedance test with cell lysis. The impedance test without cell lysis presented a better sensitivity with values of 94.87, 100, 100 and 90.48% obtained in less than 30 minutes. In addition, the intensity of the signal (in arbitrary units) for detected strains was better with the impedance test without cell lysis after 30 minutes (Mean=39.24; SDT=12.01) than with the impedance test with cell lysis after 60 minutes (Mean=15.15; SDT 8.12) (data not shown). The CarbaNP-test did not detect 8 OXA-48-producers on 39. Moreover, 4 strains were not interpretable with the CarbaNP-test because of color changes that were independent of the imipenem hydrolysis. Moreover, the detection of OXA-48 like producers was the most difficult because of the weaker carbapenemase activity of the Class D carbapenemases. This was particularly the case for the CarbaNP-test for which the interpretation of the color change from red to orange could be dependent of the reader.

TABLE 4
Comparison of the 3 tests on OXA-48 producers.

| | | Impedance test in | Impedance test in |
|---|---|---|---|
| | Carba NP-test | BEPR | salt buffer |
| Sensitivity (%) | 78.38 | 76.92 | 94.87 |
| Specificity (%) | 100 | 100 | 100 |
| PPV (%) | 100 | 100 | 100 |
| NPV (%) | 68 | 67.86 | 90.48 |

Finally, the method of the invention correctly identified the 10 strains from an external quality control from NEQAS (see Table 2 above). All nine carbapenemase-producers were detected within maximum 10 minutes and the negative strains were confirmed after 30 minutes with the impedance test without cell lysis. The same qualitative results were obtained with the CarbaNP-test but after 1 hour for the OXA-48-positive strains, the negative strains being confirmed after 2 hours.

Finally, 49 collection strains were tested in triplicate demonstrating the reproducibility of the impedance test without cell lysis in FIG. 8.

The emergence and spread of bacteria resistance to antimicrobial is a major public health concern. The early detection of beta-lactamase-producing strains and in particular carbapenemases is of the utmost importance either for antibiotherapy or for implementation of infection control measures.

As a result, the tests of the present invention were shown to be capable to detect carbapenemase-producing Enterobacteriaceae with a sensitivity and a specificity which are better that the existing CarbaNP-test. The test with cell lysis presents results that are comparable to the CarbaNP-test regarding sensitivity and specificity, but the test without cell lysis presents additional advantages comparatively to the CarbaNP-test and other methods based on the colorimetric change of an acidometric indicator. The technology of the invention indeed advantageously reduces the time to results from more than 2 hours (including with cell lysis) to 30 minutes.

The method of the invention takes its advantages from the fact that in addition to the acidification of the medium, the oxido-reduction also participates to the modification of the impedance of the polymer material coated on the electrode (in a preferred embodiment, PANI). The sensor of the invention is hence a better sensor for detecting the hydrolysis conducted by beta-lactamases, carbapenemases and/or cephalosporinases than the colorimetric or iodine indicators disclosed in the art. In a particular embodiment, the test of the invention is thus an electrochemical test permitting the measurement and the traceability of the signal, and thus represents a significant improvement, especially in the scope of accreditation process for the clinical laboratory.

In a preferred embodiment, the test is performed at room temperature, hence permitting the real-time observation of the results and avoids the requirement of an incubator. The technology of the invention also allows parallelizing the electrodes up to 384 tests which could be used for high throughput or for the screening of molecules potentially inhibiting the carbapenemases.

Further, in a preferred embodiment of the invention, cell lysis is no more required for implementing the detection test of the invention. Further the technology of the invention improves the sensitivity of the method especially for detecting OXA-48 producers which present a weak hydrolysis of the substrate. In the above disclosed experiments, the sensitivity of the detection was significantly improved (94.9% vs 78.4%) when compared to the CarbaNP-test of the prior art, especially for the detection of OXA-48. GES-6 was nevertheless not detected by the test of the invention, presumably because this very rare carbapenemase seems to be a very weak carbapenem hydrolyser for which precise enzymatic characteristics are not described yet.

Besides the carbapenems hydrolysis, the technology of the invention is perfectly suited to follow the hydrolysis of any other substrate of an enzyme activity directed to beta-lactam antimicrobial agents.

### Example 4: Reusable Electrodes

A Klebsiella pneumoniae OXA-48 strain [NEQAS1943 or PEP136] was tested 8 times on the same electrode: 4 times with imipenem, and 4 times without imipenem. The same electrode was then reuse 19 times consecutively to prove its reusability.

A full 10 μL loop of the cultured bacteria was resuspended 110 μL of lysis buffer (75 mM MgCl₂+75 mM CaCl₂). Twenty four μL of this suspension were added to 80 μL of a solution containing 0.1 mM of ZnSO₄ with or without 3 mg/mL of imipenem (Imi). Fifty μL of this latter suspension were then loaded on the electrode according to the following scheme:

First measurement including electronic reset step: −Imi/+Imi/−Imi/+Imi/−Imi/+Imi/−Imi/+Imi.

For the second measurement, the electrode was rinsed with water and reloaded according to the following alternative scheme in order to prove the total efficiency of the reset mode: +Imi/−Imi/+Imi/−Imi/+Imi/−Imi/+Imi−Imi and measured.

The electrode was then rinsed again and reloaded according to the scheme used for the first measurement and the measurement started again. This process was repeated till the same electrode was used 19 times.

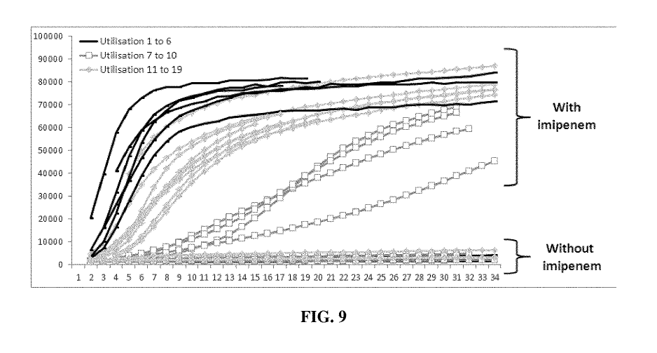

Results are shown on FIG. 9. On this figure, one curve represents the mean of 4 curves obtained in one use of the electrode (with or without imipenem).

19 curves without imipenem are flat.

Utilizations 1 to 6 measuring Klebsiella pneumoniae OXA-48 NEQAS 1943 shows the same shape.

Utilizations 7 to 10 realized with Klebsiella pneumoniae OXA-48 PEP136 incubated 48 hour (instead of 16 to 24 hour) shows weaker curves.

Utilizations 11 to 19 measuring again Klebsiella pneumoniae OXA-48 NEQAS 1943 proves correct results.

Therefore, these results demonstrate that, when the method of the invention comprises a first reset step, the electrode may be reused for at least 19 times.

### Example 5: Method with or without Negative Control

In the first generation of the BYG test, the BYG test comprises the comparison of a signal with imipenem and a signal without imipenem. When the difference between these 2 signals is “significant” (i.e. crosses a specific threshold), a strain is considered as producing an enzyme activity capable of hydrolyzing an agent comprising a beta-lactam ring, such as, for example, a carbapenemase. This means that 2 fingers (electrodes) are needed for one strain.

In a second generation of the BYG test, the comparing step is withdrawn, i.e. the method only comprises analyzing the fingers with imipenem. Indeed, the Inventors demonstrated that very valuable results may be obtained when analyzing only fingers with imipenem. This means only one electrode for one strain which reduce the cost of the electrode/analysis by 2.

Currently, on 319 Enterobacteriaceae prospectively analysed with the BYG test of the invention, when subtracting the value without imipenem from the value with imipenem, a sensitivity of 97% and a specificity of 100% were reached (only 5 Enterobacteriaceae producer of carbapenemase were not detected).

In the same time, when only looking at the signal with imipenem for the same strains, with a readjusted threshold, it was possible to maintain 100% specificity with a sensitivity of 95% (9 Enterobacteriaceae producer of carbapenemase are not detected: the 5 same isolates of the first analyses+4 additional strains).

It is hence a very unique characteristic of the method of the invention which permits to obtain a sensitivity of 95% (which is very acceptable on a clinical point of view) on a large collection of prospective strains without the need of a negative control. On the contrary, a negative control is mandatory for colorimetric technique.

1-18. (canceled)

19. A method for detecting, in a sample, a beta-lactamase activity, wherein said method is an impedance assay comprising the steps of:
(i) contacting the sample with at least one substrate of said beta-lactamase activity in at least one electrochemical cell; and
(ii) detecting an impedance variation in said electrochemical cell by collecting data points;
wherein said at least one substrate comprises a beta-lactam ring.

20. The method according to claim 19, wherein steps (i) and (ii) are performed simultaneously.

21. The method according to claim 19, wherein said beta-lactamase activity is a carbapenemase activity or a cephalosporinase activity.

22. The method according to claim 19, wherein the sample comprises a free enzyme.

23. The method according to claim 19, wherein the sample comprises a biological cell.

24. The method according to claim 19, wherein the sample comprises a bacteria.

25. The method according to claim 19, wherein the sample comprises a gram-negative bacteria selected from the group comprising enterobacterial cells and non-fermenting gram-negative bacteria cells.

26. The method according to claim 19, wherein said substrate is selected from penams, cephems, monobactams, carbapenems, carbapenams, clavams, penems, carbacephems and oxacephems or a combination thereof.

27. The method according to claim 19, wherein the first step is performed in the presence of at least one cofactor salt.

28. The method according to claim 19, wherein the first step is performed in the presence of at least one secondary salt.

29. The method according to claim 19, wherein the first step is performed in the presence of at least one cofactor salt, which is ZnSO₄, and in the presence of at least one secondary salt, selected from CaCl₂, MnCl₂, MgCl₂, NaCl or KCl or any combination selected from CaCl₂ and MnCl₂ or CaCl₂ and MgCl₂.

30. The method according to claim 19, further comprising a step of lysing the biological cell.

31. The method according to claim 19, wherein said method does not comprise a step of lysing the biological cell.

32. The method according to claim 19, being a method for identifying a beta-lactamase activity, wherein said method is further defined as comprising the steps of:
(i) contacting a sample suspected to contain said beta-lactamase activity with at least one substrate thereof in at least one electrochemical cell, with at least one possible inhibitor of said beta-lactamase activity;
(ii) contacting the sample with said at least one substrate in at least one electrochemical cell, without the said at least one possible inhibitor;
(iii) detecting an impedance variation in said electrochemical cells of steps (i) and (ii) by collecting data points; and
(iv) comparing the impedance variations detected in step (iii);
wherein said at least one substrate comprises a beta-lactam ring.

33. The method according to claim 1, being a method for screening candidate inhibitors for inhibiting an beta-lactamase activity, wherein said method is further defined as comprising the steps of:
(i) contacting a sample comprising said beta-lactamase activity with at least one substrate of said beta-lactamase activity and at least one candidate inhibitor, in at least one electrochemical cell;
(ii) contacting the sample with the said at least one substrate of said beta-lactamase activity without the said at least one candidate inhibitor;
(iii) detecting an impedance variation in said electrochemical cells of steps (i) and (ii) by collecting data points; and
(iv) comparing the impedance variations detected in step (iii);
wherein said at least one substrate comprises a beta-lactam ring.

34. A method for screening candidate beta-lactam agents that are not hydrolyzed by a beta-lactamase activity, comprising the steps of:
(i) contacting a sample comprising said beta-lactamase activity (either within a biological cell or in a free form) with at least one candidate beta-lactam agent, in at least one electrochemical cell;
(ii) contacting the sample with a known substrate of said beta-lactamase activity;
(iii) detecting an impedance variation in said electrochemical cells of steps (i) and (ii) by collecting data points; and
(iv) comparing the impedance variations detected in step (iii;
wherein said at least one candidate anti-microbial agent comprises a beta-lactam ring.

35. A system for detecting, in a sample, a beta-lactamase activity by measuring impedance of an a working electrode, the system comprising:
a multiplexer comprising at least a 499 kΩ resistor and infinite resistor,
a working electrode made of an electro-conductive solid polymer transducer and coated with polyaniline;
an input to receive an input signal indicative of the potential to be applied between said working electrode and a reference electrode; and
an output to transmit an output signal indicative of the magnitude of the current flowing between a counter electrode and said working electrode;
said working and reference electrodes being adapted to be immerged into the sample or to be loaded with the sample;
a digital processor connected to a digital to analog converter for generating the input signal; and to an analog to digital converter for receiving at least one data point, which is a digital value;
a computer collecting at least 80 data points, and calculating contiguous integrals of the data points in order to recover parameters summed to correspond to a global conductance.

36. System according to claim 35, wherein the polyaniline coated electrode is reusable.

37. System according to claim 35, wherein the working electrode is coated with polyaniline and at least one substrate of a beta-lactamase activity.

38. The method according to claim 19, wherein the step of detecting an impedance variation comprises:
collecting exchanged charges in the form of data points in the electrochemical cell using a system comprising:
a multiplexer comprising at least a 499 kΩ resistor and infinite resistor,
a working electrode made of an electro-conductive solid polymer transducer and coated with polyaniline;
an input to receive an input signal indicative of the potential to be applied between said working electrode and a reference electrode; and
an output to transmit an output signal indicative of the magnitude of the current flowing between a counter electrode and said working electrode;
said working and reference electrodes being adapted to be immerged into the sample or to be loaded with the sample;
a digital processor connected to a digital to analog converter for generating the input signal; and to an analog to digital converter for receiving at least one data point, which is a digital value;
a computer collecting at least 80 data points, and calculating contiguous integrals of the data points in order to recover parameters summed to correspond to a global conductance; and
calculating contiguous integrals of the data points and summing the integrals to obtain global conductance.
